(19) United States
(12) Patent Application Publication
(10) Pub. No.: US 2025/0179128 A1
(43) Pub. Date: Jun. 5, 2025

(54) RECOMBINANT SPIDER SILK PROTEINS

(71) Applicants: Anna RISING, Uppsala (SE); Benjamin SCHMUCK, Uppsala (SE); Jan JOHANSSON, Stockholm (SE)

(72) Inventors: Anna RISING, Uppsala (SE); Benjamin SCHMUCK, Uppsala (SE); Jan JOHANSSON, Stockholm (SE)

(21) Appl. No.: 18/843,384

(22) PCT Filed: Mar. 3, 2023
(86) PCT No.: PCT/SE2023/050191

§ 371 Date: Sep. 3, 2024

(30) Foreign Application Priority Data
Mar. 4, 2022 (SE) 2250294-2

(51) Int. Cl. C07K 14/435; C12N 15/70; C12P 21/02

(52) U.S. Cl. CPC C07K 14/43518; C12N 15/70; C12P 21/02; C12N 2800/101

(57) ABSTRACT

A recombinant spider silk protein comprises an NT domain, a REP domain and a CT domain. The REP domain comprises a set of domains according to the formula pA₁-pG-pA₂. pG represents a glycine-rich domain and pA₁ and pA₂ represent alanine-rich domains. One of pA₁ and pA₂ is a poly-alanine domain and the other of pA₁ and pA₂ is a poly-alanine domain having every third or fourth alanine residue replaced by an isoleucine residue or a valine residue. Spider silk spun from the recombinant spider silk proteins of the invention have improved mechanical properties.

### TECHNICAL FIELD

The present invention generally relates to recombinant spider silk proteins, and in particular such recombinant spider silk proteins that can produce silk fibers having improved mechanical properties.

### BACKGROUND

Spiders can spin seven types of silk, each with unique mechanical properties, produced in different glands; major ampullate, minor ampullate, flagelliform, tubuliform, aciniform, aggregate and piriform.

These silks are made of silk proteins that are named according to their primary gland of expression, major ampullate spider silk protein (MaSp), minor ampullate spider silk protein (MiSp), flagelliform spider silk protein (FISp), tubuliform spider silk protein (TuSp), aciniform spider silk protein (AcSp), aggregate spider silk protein (AgSp) and piriform spider silk protein (PiSp), respectively. All spider silk proteins, also referred to as spidroins in the art, have an N-terminal (NT) domain, an extensive repetitive region (REP), and a C-terminal (CT) domain. The mechanical properties are believed to be dictated by the REP domain (Guerette et al., Science 11:112-115 (1996). The most extensible fiber, the flagelliform silk, is mainly made from spider silk proteins (FISps) that carry a Pro-rich REP region, which is predicted to form spring-like structures. The strongest fiber, the major ampullate silk, also referred to as dragline, is mainly composed of spider silk proteins (MaSps) that carry a repeat region of iterated Gly-rich and poly-Ala repeats. The tensile strength of the major ampullate silk is derived from the MaSp poly-Ala blocks that form β-sheet crystals in the silk fiber, while the Gly-rich parts mediate the fiber's extensibility (Bratzel et al., J Mech Behav Biomed Mater. 7:30-40 (2012); Liu et. al, Adv Funct Mater. 26:5534-5541 (2016); Keten et al., Nat Mater. 9:359-367 (2010). The MaSp silk is the toughest natural fiber known (around 150 MJ/m³) (Gosline et al., J Exp Biol 202:3295-3303 (1999); Blackledge et al., J Exp Biol 209:2452-2461 (2006)).

A recombinant spider silk protein having improved solubility in water and thereby allowing scalable production at high yields is known in the art as NT2RepCT (WO 2018/002216; Andersson et al., Nat Chem Biol 11:309-315 (2017). NT2RepCT contains a His6-tag, a NT domain from Euprosthenops australis MaSp1, two Gly-rich and poly-Ala tandem repeats from E. australis (2Rep) and a CT domain from Araneus ventricosus MiSp.

Johansson and Rising, ACS Nano 15:1952-1959 (2021) discloses a structural-biology-based approach for engineering artificial spidroins to produce biomimetic silk fibers with improved mechanical properties.

There is, though, still a need for a recombinant spider protein capable of producing silk fibers having improved mechanical properties.

### SUMMARY

It is a general objective to provide a recombinant spider silk protein capable of producing silk fibers having improved mechanical properties.

This and other objectives are met by embodiments of the present invention.

The present invention is defined in the independent claims. Further embodiments of the invention are defined in the dependent claims.

An aspect of the invention relates to a recombinant spider silk protein comprising an N-terminal (NT) domain, a repetitive region (REP) domain and a C-terminal (CT) domain. The REP domain comprises a set of domains according to the formula pA₁-pG-pA₂. pG represents a glycine-rich domain and pA₁ and pA₂ represent alanine-rich domains. One of pA₁ and pA₂ is a poly-alanine domain and the other of pA₁ and pA₂ is a poly-alanine domain having every third or fourth alanine residue replaced by an isoleucine residue or a valine residue.

Further aspects of the invention relate to a silk fiber made of the recombinant spider silk protein according to above, a synthetic material comprising the silk fiber according to above, a nucleic acid molecule encoding the recombinant spider silk protein according to above, an expression vector comprising the nucleic acid molecule according to above, and a host cell comprising the expression vector according to above.

An additional aspect of the invention relates to a method for producing a silk fiber. The method comprises extruding a spinning dope comprising the recombinant spider silk protein according to above into an aqueous buffer having an acidic pH to induce polymerization of the recombinant spider silk protein into a silk fiber. The method also comprises isolating the silk fiber from the aqueous buffer.

The recombinant spider silk proteins of the invention can be spun into silk fibers having very high tensile strength and strain at break and a toughness equal to native dragline silks. The recombinant spider silk proteins of the invention can also be produced in high yields and concentrated to high concentration for producing spinning dopes that are suitable for production of silk fibers.

### BRIEF DESCRIPTION OF THE DRAWINGS

The embodiments, together with further objects and advantages thereof, may best be understood by making reference to the following description taken together with the accompanying drawings, in which:

FIG. 1. Schematic representation of the designed constructs. A) NT2RepCT (A₁₅-A₁₄) is composed of an N-terminal domain (NT; PDB: 4FBS), a repeat region with two poly-Ala blocks, and a C-terminal domain (CT, PDB 3LR2). Both subunits of the soluble NT2RepCT dimer are shown (one is shaded). B) Protein sequence alignment of the repetitive region from A₁₅-A₁₄ and engineered constructs thereof. Note that all constructs contain NT, a repeat part and CT. Substitutions in the poly-Ala blocks are indicated. Sequences presented in B) are found in SEQ ID NO: 1-16.

FIG. 2. Rosetta energy profiles of A) A₁₅-A₁₄ and (A₃I)₃-A₁₄ (profiles for all designed proteins are found in Table 1). Bars show Rosetta energies for moving hexapeptides (indicated at the first residue of each hexapeptide), dark gray bars indicate Rosetta energies equal or below-23 kcal/mol (dashed line). Light gray bars indicate Rosetta energies above the threshold and are unlikely to form steric zippers (https://services.mbi.ucla.edu/zipperdb/). B) Bars indicate the Rosetta energy of the hexapeptide with the lowest predicted energy from A₁₅-A₁₄ and the engineered mini-spidroins (all hexapeptides are shown in Table 1). C) Hypothetical zipper structure of two β-sheets composed of hexapeptides AAAAAA (SEQ ID NO: 17) from A₁₅-A₁₄ and AIAAAI (SEQ ID NO: 24) derived from (A₃I)₃-A₁₄, respectively.

FIG. 3. CD spectroscopy of purified engineered mini-spidroins. A) Initial spectra at 20° C. and B) molar ellipticity measured at 222 nm from 20° C. to 90° C. was converted to fraction natively folded (%) and then normalized. CD spectroscopy of different constructs C) heated to 90° C. and D) after cooling to 20° C.

FIG. 4. Mechanical properties of spinnable engineered mini-spidroins in comparison with A₁₅-A₁₄. A) Photographs of spun fibers, B) strength, C) strain at break, D) toughness modulus, dashed line indicates toughness modulus of a native dragline silk and E) representative stress-strain curves. Note, (A₃T)₃-(A₃T)₃ and (A₃V)₃-(A₃V)₃ have very low strains, a zoomed in graph showing these can be found in FIG. 7A. Whiskers show standard deviation. * p<0.05; ** p<0.01; *** p<0.001; **** p<0.0001

FIG. 5. FTIR spectroscopy of engineered fibers. Normalized and baseline-subtracted absorbance spectrum in the amide I region of A) A₁₅-A₁₄, (A₃V)₃-(A₃V)₃, (A₃V)₃-A₁₄, (A₃T)₃-(A₃T)₃ and B) A₁₅-A₁₄, (A₃I)₃-A₁₄, A₁₅-(A₃I)₃ and (A₂I)₄-A₁₄. C) Percent secondary structure content determined by co-fitting the absorbance spectrum and the second derivative. Horizontal line indicates β-sheet content of A₁₅-A₁₄.

FIG. 6. Solid-state NMR ¹³C-¹³C correlation spectra (aliphatic region) of A₁₅-A₁₄ fibers (dark gray) (A₃I)₃-A₁₄ fibers (light gray). The Cα/Cβ correlations of Ala and Ile in α-helical and β-sheet conformation are indicated.

FIG. 7. Mechanical properties of spinnable constructs continued. A) Zoom in on representative stress-strain curves. Full stress-strain curves can be found in FIG. 4E. B) Young's modulus and C) diameter of the fiber.

FIG. 8. Extrusion of A₁₅-A₁₄ or (A₃I)₃-A₁₄ at 17 or 35 μl/min through a tapered metal nozzle with an orifice diameter of 150 μm.

### DETAILED DESCRIPTION

The present invention generally relates to recombinant spider silk proteins, and in particular such recombinant spider silk proteins that can produce silk fibers having improved mechanical properties.

The spider silk proteins of the invention are recombinant or engineered spider silk proteins, i.e., are artificial and non-naturally occurring spider silk proteins. The recombinant spider silk proteins are preferably in the form of isolated recombinant spider silk proteins. The recombinant spider silk proteins of the invention can produce silk fibers having improved mechanical properties as compared to NT2RepCT (WO 2018/002216; Andersson et al., Nat Chem Biol 11:309-315 (2017). In more detail, silk fibers produced from recombinant spider silk proteins have significantly higher strength, strain at break and toughness modulus as compared to NT2RepCT.

Another significant advantage of the recombinant spider silk proteins of the present invention is that they can be produced at high yield and concentration. Such high yields and concentrations are advantageous when preparing a spinning dope that is used to spin silk fibers. Generally, such a spinning dope should contain a very high concentration of the spider silk protein in order to facilitate production of silk fibers.

An aspect of the invention therefore relates to a recombinant spider silk protein comprising an N-terminal (NT) domain, a repetitive region (REP) domain and a C-terminal (CT) domain. According to the invention, the REP domain comprises a set of domains according to the formula pA₁-pG-pA₂. pG represents a glycine-rich (G-rich or Gly-rich) domain and pA₁ and pA₂ represent alanine-rich (A-rich or Ala-rich) domains. According to the invention, one of pA₁ and pA₂ is a poly-alanine domain and the other of pA₁ and pA₂ is a poly-alanine domain having every third or fourth alanine (A or Ala) residue replaced by an isoleucine (I or Ile) residue or a valine (V or Val) residue.

Spider silk proteins, also referred to as spidroins, are composed of an NT domain, a REP domain and a CT-domain. The terminal domains are important for the solubility of the spider silk proteins during storage and regulate the assembly of the spider silk proteins into a solid fiber. The REP domain of most major ampullate spidroins (MaSps) contains up to 100 tandem repeats of poly-alanine blocks and glycine-rich motifs. In the soluble dope, the spider silk proteins are mostly in random coil and helical conformations, whereas the solid silk fiber contains nano-sized crystals made up of stacked anti-parallel β-sheets embedded in amorphous structures. This heterogenous structure of the silk fiber is important as the β-sheet crystals confer the strength, while the amorphous structures confer extensibility to the fiber. The amorphous matrix, containing β-turns and ordered structures with conformational similarities to collagen and poly-proline helices, are dominated by the glycine-rich regions. The β-sheets, formed by the poly-alanine blocks, orient with the β-strands parallel to the fiber axis, and the alanine side chain of a given β-strand fill the space close to an α-carbon in a neighboring β-strand, analogous to a tightly packed steric zipper.

There are two main strategies for producing artificial silk fibers; one being expression of insoluble spider silk proteins with subsequent solubilization and fiber processing using organic solvents, and another being a biomimetic approach involving only aqueous solutions throughout the purification and spinning procedures and in which the molecular mechanisms and triggers for fiber formation are replicated. The first approach enables expression of large spider silk proteins that can be spun into silk fibers with high tensile strength, but the protein yields are far from what is required for industrial production (Bowen et al., Biomacromolecules 19:3853 (2018); Edlund et al., New Biotechnology 42:12 (2018)). Using the second approach, small spider silk proteins, often referred to as mini-spidroins, composed of an NT-domain, a short REP domain generally consisting of two poly-alanine/glycine-rich domains and a CT-domain, have been developed. Such mini-spidroins are extremely water-soluble and can be spun into silk fibers using biomimetic spinning set-ups. Moreover, one of these mini-spidroins, NT2RepCT, can be produced at a yield of 14.5 g/L in bioreactor cultivations, which vouch for economically feasible bulk production (Edlund et al., New Biotechnology 42:12 (2018); Schmuck et al., Materials Today 50:16 (2021)). Silk fibers spun from NT2RepCT are superior compared to previously published as-spun silk fibers, but still, the silk fibers only reach about 15% of the native silk fiber's tensile strength (Gosline et al., Journal of Experimental Biology 202:3295 (1999); Andersson et al., Nature Chemical Biology 13:262 (2017). NMR spectroscopy revealed that the two poly-alanine domains of the mini-spidroin are in an α-helical conformation in the soluble state and convert to β-sheet conformation in the as-spun wet fiber. However, upon drying the silk fiber, the poly-alanine domains are transitioning back to α-helical conformation (Otikovs et al., Angew Chem Int Ed Engl 56:12571 (2017)), which could explain the inferior mechanical properties of dried NT2RepCT fibers compared to the native silk fiber.

The recombinant spider silk proteins of the present invention improve the mechanical properties of silk fibers by increasing the β-strand propensity and inter-β-sheet interactions of the poly-alanine domains. Notably, alanine residues have a low propensity to form β-strands, whereas more hydrophobic residues like valine, cysteine, isoleucine, and phenylalanine show a higher β-strand propensity, and, thus, could be considered better candidates for forming stable β-sheets in the silk fiber. However, being secretory proteins, the spider silk proteins need to pass through the translocon when produced by the gland epithelium. If the nascent polypeptide chain contains segments that are rich in valine, isoleucine, cysteine, or phenylalanine the translocon will mediate insertion into the endoplasmic reticulum (ER) membrane, and, thus, any spidroin segment rich in these amino acid residues would be trapped in the cell. In fact, alanine is the most hydrophobic residue that allows passage through the translocon, which suggests that the spider silk proteins have evolved to optimize hydrophobicity in their β-sheet forming segments to the extent possible for a secretory protein. Intracellular expression in prokaryotes will bypass the restrictions imposed by the secretory pathway that native spider silk proteins must adhere to since translation and accumulation of the target protein take place in the cytosol.

The tensile strength of silk fibers is conferred by poly-alanine stretches that are zipped together by tight side chain packing in β-sheet crystals. Spider silk proteins are secreted so they must be void of long stretches of hydrophobic residues, since such segments get inserted into the ER membrane. At the same time, hydrophobic residues have high β-strand propensity and can mediate tight inter-β-sheet interactions, features that are attractive for generation of strong artificial silks. The recombinant spider silk proteins of the invention are predicted to more avidly form stronger β-sheets than the wildtype protein by selective replacement of alanine residues by isoleucine or valine residues in one of the poly-alanine domains.

As is further shown in the Example section, replacement of alanine residues in alanine-rich domains with threonine (T) either do not express well or result in silk fibers with significantly less extensibility (strain at break) and toughness as compared to the spider silk proteins of the invention. This was true even though threonine is branched at the β-carbon and, hence, should favor β-strand conformation in the silk protein.

Furthermore, experimental data as presented herein indicates that exchanging more than every third or fourth alanine residue, such as every second alanine residue or indeed every alanine residue, with an isoleucine or valine residue led to insoluble spider silk proteins or very low amounts of soluble spider silk proteins even if performed in only one of the two alanine-rich domains. Correspondingly, exchanging every seventh alanine residue caused protein aggregation and could thereby not be spun into silk fibers.

Experimental data herein further shows that replacing alanine residues in both alanine-rich domains, i.e., in both pA₁ and pA₂, led to very fragile silk fibers or silk fibers having inferior mechanical properties in terms of low strain at break and low toughness.

Accordingly, improved mechanical properties of silk fibers are obtained when one of the alanine-rich domains of the REP domain is a poly-alanine domain and the other of the alanine-rich domains of the REP domain is a poly-alanine domain having every third or fourth alanine residue replaced by an isoleucine residue or a valine residue.

The alanine-rich domain that is a poly-alanine domain thereby comprises a plurality of alanine residues and these residues are not interposed by any other amino acid residues. Hence, this poly-alanine domain preferably consists only of alanine residues.

In an embodiment, the one of pA₁ and pA₂ comprises, preferably consists of, an amino acid sequence Aₘ. In this embodiment, m is an integer selected within an interval of from 7 up to 18, preferably within an interval of from 10 up to 17, and more preferably within an interval of from 14 up to 16.

Hence, in this embodiment, one of the alanine-rich domains of the REP domain comprises, preferably consists of, a sequence of alanine residues, in more detail m such consecutive alanine residues. The length of this poly-alanine domain is from 7 up to 18 alanine residues and preferably from 10 up 17 alanine residues. In particular preferred embodiments, the poly-alanine domain has a length from 14 up to 16 alanine residues, such as 14 alanine residues, 15 alanine residues or 16 alanine residues, and more preferably 14 or 15 alanine residues.

In an embodiment, the length of the other alanine-rich domain, i.e., the poly-alanine domain having alanine residues replaced by isoleucine or valine residues, is preferably from 8 up to 18 amino acid residues, preferably from 10 up to 17 amino acid residues and more preferably from 14 up to 16 amino acid residues, such as 14 amino acid residues, 15 amino acid residues or 16 amino acid residues, and more preferably 14 or 15 amino acid residues.

In an embodiment, the other of pA₁ and pA₂ is a poly-alanine domain having every third or fourth alanine residue replaced by an isoleucine residue or a poly-alanine domain having every third or fourth alanine residue replaced by a valine residue. Hence, in this embodiment, the other of pA₁ and pA₂ comprises, preferably consists of, alanine and isoleucine residues or comprises, preferably consists of, alanine and valine residues.

It is, though, possible to have a poly-alanine domain comprising, preferably consisting of, alanine, isoleucine and valine residues. In such an embodiment, at least one of every third or fourth alanine residue of the poly-alanine domain is replaced by an isoleucine residue and at least one of every third or fourth alanine residue of the poly-alanine domain is replaced by a valine residue.

In an embodiment, the other of pA₁ and pA₂ is a poly-alanine domain having every fourth alanine residue replaced by an isoleucine residue or a valine residue. Experimental data as shown herein shows that recombinant spider silk proteins having one of the alanine-rich domains in the form of a poly-alanine domain having every fourth alanine residue replaced by an isoleucine residue or a valine residue produced silk fibers with improved mechanical properties in terms of strength, strain at break and toughness as compared to recombinant spider silk proteins having one of the alanine-rich domains in the form of a poly-alanine domain having every third alanine residue replaced by an isoleucine residue or a valine residue.

In a particular embodiment, the other of pA₁ and pA₂ is a poly-alanine domain having every fourth alanine residue replaced by an isoleucine residue. Experimental data shows that recombinant spider silk proteins having an alanine-rich domain with replacements of alanine residues with isoleucine residues produced silk fibers with higher strength, strain at break, and toughness as compared to corresponding recombinant spider silk proteins having an alanine-rich domain with replacements of alanine residues with valine residues.

Hence, in an embodiment, the other of pA₁ and pA₂ is a poly-alanine domain having every third or fourth alanine residue replaced by an isoleucine residue, preferably every fourth alanine residue replaced by an isoleucine residue.

In an embodiment, the other of pA₁ and pA₂ comprises, preferably consists of, an amino acid sequence selected from the group consisting of (A₃I)ₙAₚ, Aₚ(IA₃)ₙ, (A₃V)ₙAₚ and Aₚ(VA₃)ₙ. In this embodiment, n is an integer selected within an interval of from 2 up to 4, p=m−n, and m is an integer selected within an interval of from 8 up to 18.

In a particular embodiment, n is 3.

In a particular embodiment, m is an integer selected within an interval of from 10 up to 18, preferably within an interval of from 14 up to 16, and more preferably m is 14 or 15.

Currently, preferred amino acid sequences of the other of pA₁ and pA₂ comprises, preferably consists of, AAAIAAAIAAAIAA (SEQ ID NO: 43), AAAIAAAIAAAIAAA (SEQ ID NO: 44), AAIAAAIAAAIAAA (SEQ ID NO: 45), AAAVAAAVAAAVAA (SEQ ID NO: 46), AAAVAAAVAAAVAAA (SEQ ID NO: 47), and AAVAAAVAAAVAAA (SEQ ID NO: 48).

In a particular embodiment, the other of pA₁ and pA₂ comprises, preferably consists of, an amino acid sequence selected from the group consisting of (A₃I)ₙAₚ and Aₚ(IA₃)ₙ. Currently preferred amino acid sequences according to this particular embodiment are AAAIAAAIAAAIAA (SEQ ID NO: 43), AAAIAAAIAAAIAAA (SEQ ID NO: 44), and AAIAAAIAAAIAAA (SEQ ID NO: 45).

In a preferred embodiment, the other of pA₁ and pA₂ comprises, preferably consists of, an amino acid sequence according to (A₃I)ₙAₚ. Currently preferred amino acid sequences according to this embodiment are AAAIAAAIAAAIAA (SEQ ID NO: 43) and AAAIAAAIAAAIAAA (SEQ ID NO: 44).

The REP domain comprises alternating glycine-rich domain(s) and alanine-rich domains. In an embodiment, the REP domain comprises a set of domain according to a formula pA₁-pG-pA₂, pA₁-pG₁-pA₂-pG₂, pG₁-pA₁-pG₂-pA₂ or pG₁-pA₁-pG₂-pA₂-pG₃. In this embodiment, pG, pG₁, pG₂ and pG₃ represent glycine-rich domains. Hence, in this embodiment, the REP domain comprises, preferably consists of, two alanine-rich domains and one, two or three glycine-rich domains. Furthermore, the alanine-rich domains and the glycine-rich domain(s) are alternating domains in the REP domain.

In a particular embodiment, the REP domain comprises two alanine-rich domains, preferable consists of two-alanine-rich domains, and one or more, preferably one to three, and more preferably two or three, and even more preferably three glycine-rich domains.

In a particular embodiment, the REP domain comprises a set of domains according to a formula pA₁-pG₁-pA₂-pG₂, pG₁-pA₁-pG₂-pA₂ or pG₁-pA₁-pG₂-pA₂-pG₃. In this particular embodiment, the REP domain comprises, preferably consists of, two alanine-rich domains and two or three glycine-rich domains. In a preferred embodiment, the REP domain comprises a set of domains according to a formula pG₁-pA₁-pG₂-pA₂-pG₃.

In an embodiment, the REP domain consists of pA₁-pG-pA₂, pA₁-pG₁-pA₂-pG₂, pG₁-pA₁-pG₂-pA₂ or pG₁-pA₁-pG₂-pA₂-pG₃. In a particular embodiment, the REP domain consists of pA₁-pG₁-pA₂-pG₂, pG₁-pA₁-pG₂-pA₂ or pG₁-pA₁-pG₂-pA₂-pG₃. In a preferred embodiment, the REP domain consists of pG₁-pA₁-pG₂-pA₂-pG₃.

In an embodiment, the REP domain comprises a set of domains according to a formula selected from the group consisting of (A₃I)₃A₃-pG-A₁₄, A₁₅-pG-(A₃I)₃A₂, (A₃V)₃A₃-pG-A₁₄, A₁₅-pG-(A₃V)₃A₂, (A₃I)₃A₃-pG₁-A₁₄-pG₂, A₁₅-pG₁-(A₃I)₃A₂-pG₂, pG₁-(A₃I)₃A₃-pG₂-A₁₄, pG₁-A₁₅-pG₂-(A₃I)₃A₂, pG₁- (A₃I)₃A₃-pG₂-A₁₄-pG₃, pG₁-A₁₅-pG₂-(A₃I)₃A₂-pG₃, (A₃V)₃A₃-pG₁-A₁₄-pG₂, A₁₅-pG₁-(A₃V)₃A₂-pG₂, pG₁-(A₃V)₃A₃-pG₂-A₁₄, pG₁-A₁₅-pG₂-(A₃V)₃A₂, pG₁-(A₃V)₃A₃-pG₂-A₁₄-pG₃ and pG₁-A₁₅-pG₂-(A₃V)₃A₂-pG₃, preferably selected from the group consisting of (A₃I)₃A₃-pG₁-A₁₄-pG₂, A₁₅-pG₁-(A₃I)₃A₂-pG₂, pG₁-(A₃I)₃A₃-pG₂-A₁₄, pG₁-A₁₅-pG₂-(A₃I)₃A₂, pG₁-(A₃I)₃A₃-pG₂-A₁₄-pG₃, pG₁-A₁₅-pG₂-(A₃I)₃A₂-pG₃, (A₃V)₃A₃-pG₁-A₁₄-pG₂, A₁₅-pG₁-(A₃V)₃A₂-pG₂, pG₁-(A₃V)₃A₃-pG₂-A₁₄, pG₁-A₁₅-pG₂-(A₃V)₃A₂, pG₁-(A₃V)₃A₃-pG₂-A₁₄-pG₃ and pG₁-A₁₅-pG₂-(A₃V)₃A₂-pG₃.

In a particular embodiment, the REP domain comprises a set of domains according to a formula selected from the group consisting of (A₃I)₃A₃-pG₁-A₁₄-pG₂, A₁₅-pG₁-(A₃I)₃A₂-pG₂, pG₁-(A₃I)₃A₃-pG₂-A₁₄, pG₁-A₁₅-pG₂-(A₃I)₃A₂, pG₁-(A₃I)₃A₃-pG₂-A₁₄-pG₃ and pG₁-A₁₅-pG₂-(A₃I)₃A₂-pG₃.

In a preferred embodiment, the REP domain comprises a set of domains according to a formula selected from the group consisting of pG₁-(A₃I)₃A₃-pG₂-A₁₄-pG₃ and pG₁-A₁₅-pG₂-(A₃I)₃A₂-pG₃. In a currently preferred embodiment, the REP domain comprises a set of domains according to a formula selected from the group consisting of pG₁-(A₃I)₃A₃-pG₂-A₁₄-pG₃.

In an embodiment, each of pG, pG₁, pG₂ and pG₃ comprises, preferably consists of, an amino acid sequence selected from the group consisting of GRGQGGYGQGSGGN (SEQ ID NO: 49), GQGGQGGYGRQSQGAGS (SEQ ID NO: 50) and GSGQGGYGGQGQGGYGQS (SEQ ID NO: 51).

In a particular embodiment, pG₁ comprises, preferably consists, of an amino acid sequence according to SEQ ID NO: 49, pG₂ comprises, preferably consists, of an amino acid sequence according to SEQ ID NO: 50 and pG₃ comprises, preferably consists, of an amino acid sequence according to SEQ ID NO: 51.

Examples of REP domains of the recombinant spider silk proteins of the invention are presented below.

(A₃I)₃-A₁₄,
SEQ ID NO: 13
GRGQGGYGQGSGGNAAAIAAAIAAAIAAAGQGGQGGYGRQSQGAGSAAAA
AAAAAAAAAAGSGQGGYGGQGQGGYGQS
A₁₅-(A₃I)₃,
SEQ ID NO: 14
GRGQGGYGQGSGGNAAAAAAAAAAAAAAAGQGGQGGYGRQSQGAGSAAAI
AAAIAAAIAAGSGQGGYGGQGQGGYGQS
(A₃V)₃-A₁₄,
SEQ ID NO: 8
GRGQGGYGQGSGGNAAAVAAAVAAAVAAAGQGGQGGYGRQSQGAGSAAAA
AAAAAAAAAAGSGQGGYGGQGQGGYGQS
A₁₅-(A₃V)₃,
SEQ ID NO: 52
GRGQGGYGQGSGGNAAAAAAAAAAAAAAAGQGGQGGYGRQSQGAGSAAAV
AAAVAAAVAAGSGQGGYGGQGQGGYGQS

The recombinant spider silk protein preferably comprises the REP domain arranged between the NT domain and the CT domain. Hence, the recombinant preferably has the general formula NT-REP-CT.

As is further described herein, the recombinant spider silk protein may also comprise other amino acid sequences than the NT, REP and CT domains, including optional N-terminal and/or C-terminal tags and/or optional linkers. Hence, in an embodiment, the recombinant spider silk protein has the general formula (X)-NT-(L1)-REP-(L2)-CT-(Y). In this embodiment, X represents an optional N-terminal tag, Y represents an optional C-terminal tag, L1 represents an optional first linker and L2 represents an optional second linker.

As mentioned above, the recombinant spider silk proteins of the invention may contain additional amino acid sequences or domains in addition to the NT domain, the REP domain and CT domain. Such additional domains are then preferably attached to the N-terminus of the NT domain of the recombinant spider silk protein and/or to the C-terminus of the CT domain of the recombinant spider silk protein, i.e., X-NT-REP-CT, NT-REP-CT-Y or X-NT-REP-CT-Y, and/or could be provided between the NT and REP domains and/or between the REP and CT domains, i.e., NT-L1-REP-CT, NT-REP-L2-CT or NT-L1-REP-L2-CT. It is also possible to combine the N-terminal and/or C-terminal tags, X, Y, with linkers, such as X-NT-L1-REP-CT, X-NT-REP-L2-CT, X-NT-L1-REP-L2-CT, NT-L1-REP-CT-Y, NT-REP-L2-CT-Y, NT-L1-REP-L2-CT-Y, X-NT-L1-REP-CT-Y, X-NT-REP-L2-CT-Y or X-NT-L1-REP-L2-CT-Y.

Illustrative, but non-limiting, examples of such additional domains X, Y are affinity tags, solubilization tags, chromatography tags, epitope tags, fluorescence tags, signal peptides or sequences, etc.

Examples of domains facilitating purification include various affinity tags, such as chitin binding protein (CBP), maltose binding protein (MBP), hemagglutinin tag, Strep-tag, glutathione-S-transferase (GST), and poly (His) tags, such as His6 tag; solubilization tags, such as thioredoxin (TRX) and poly (NANP); chromatography tags, such as FLAG-tag; epitope tags, such as ALFA-tag, V5-tag, Myc-tag, HA-tag, Spot-tag, T7-tag and NE-tag; and fluorescence tags, such as GFP.

Illustrative examples of linkers that could be used between the NT and REP domains and/or between the REP and CT domain are various GS or GNS linkers and other peptide linkers. Such linkers may be beneficial to provide a short distance between the NT and REP domains and/or between the REP and CT domain and thereby reduce the risk of any steric hindrance between the linked domains. The optional linker may be very short, such as GS or GNS, or up to some tens of amino acids, preferably no more than 20 amino acids, and more preferably no more than 15 amino acids.

The NT-domains of spider silk proteins are thought to improve the solubility of the spider silk protein and thereby enabling very high protein concentrations in the spinning dope. Furthermore, the pH dependent dimerization of the NT-domains is an important factor in allowing rapid polymerization of the spinning dope.

Some CT-domains of spider silk proteins do not exhibit a pH-sensitive solubility (Hedhammar et al., Biochemistry 47 (11): 3407-3417 (2008), but in general most CT-domains having several charged amino acid residues are in fact highly soluble and have a pH dependent solubility (Andersson et al., PLOS Biology 12 (8): e1001921 (2014)).

The recombinant spider silk protein of the invention could use various combinations of NT-domain and CT-domain together with the REP domain to form a recombinant spider silk protein that is spinnable into a silk fiber.

Illustrative, but non-limiting, examples of NT-domains that could be used according to the present invention are listed in Table 2 in US 2019/0248847, the teaching of which regarding NT-domains is hereby incorporated by reference.

In a preferred embodiment, the NT domain of the recombinant spider silk protein is derived from the NT domain of Euprosthenops australis MaSp1.

In a particular embodiment, the NT domain comprises, preferably consists of SEQ ID NO: 53.

Illustrative, but non-limiting, examples of CT-domains that could be used according to the present invention are listed in Table 1 in US 2019/0248847, the teaching of which regarding CT-domains is hereby incorporated by reference.

In a preferred embodiment, the CT domain of the recombinant spider silk protein is derived from the CT domain of Araneus ventricosus MiSp.

In a particular embodiment, the CT domain comprises, preferably consists of SEQ ID NO: 54.

In an embodiment, the recombinant spider silk protein comprises, preferably consists of, an NT domain comprising, preferably consisting of, SEQ ID NO: 53, a REP domain comprising, preferably consisting of, an amino acid sequence selected from the group consisting of SEQ ID NO: 8, 13, 14 and 52 and a CT domain comprising, preferably consisting of, SEQ ID NO: 54.

In an example, the recombinant spider silk protein comprises, preferably consists of, an NT domain comprising, preferably consisting of, SEQ ID NO: 53, a REP domain comprising, preferably consisting of, SEQ ID NO: 13, and a CT domain comprising, preferably consisting of, SEQ ID NO: 54. Such a recombinant spider silk protein is presented in SEQ ID NO: 55, in SEQ ID NO: 56 with linkers between NT and REP domains and between REP and CT domains, in SEQ ID NO: 57 with an N-terminal His tag and in SEQ ID NO: 39 with His tag and linkers.

Another example of recombinant spider silk protein of the present invention comprises, preferably consists of, an NT domain comprising, preferably consisting of SEQ ID NO: 53, a REP domain comprising, preferably consisting of SEQ ID NO: 14, and a CT domain comprising, preferably consisting of SEQ ID NO: 54. Such a recombinant spider silk protein is presented in SEQ ID NO: 58, in SEQ ID NO: 59 with linkers between NT and REP domains and between REP and CT domains, in SEQ ID NO: 60 with an N-terminal His tag and in SEQ ID NO: 40 with His tag and linkers.

A further example of recombinant spider silk protein of the present invention comprises, preferably consists of, an NT domain comprising, preferably consisting of, SEQ ID NO: 53, a REP domain comprising, preferably consisting of, SEQ ID NO: 8, and a CT domain comprising, preferably consisting of, SEQ ID NO: 54. Such a recombinant spider silk protein is presented in SEQ ID NO: 61, in SEQ ID NO: 62 with linkers between NT and REP domains and between REP and CT domains, in SEQ ID NO: 63 with an N-terminal His tag and in SEQ ID NO: 34 with His tag and linkers.

Yet another example of recombinant spider silk protein of the present invention comprises, preferably consists of, an NT domain comprising, preferably consisting of, SEQ ID NO: 53, a REP domain comprising, preferably consisting of, SEQ ID NO: 52, and a CT domain comprising, preferably consisting of, SEQ ID NO: 54. Such a recombinant spider silk protein is presented in SEQ ID NO: 64, in SEQ ID NO: 65 with linkers between NT and REP domains and between REP and CT domains, in SEQ ID NO: 66 with an N-terminal His tag and in SEQ ID NO: 67 with His tag and linkers.

An embodiment relates to a recombinant spider silk protein comprising an NT domain, a REP domain and a CT domain. According to the invention, the REP domain comprises a set of domains according to the formula pA₁-pG-pA₂. pG represents a glycine-rich domain and pA₁ and pA₂ represent alanine-rich domains. According to the invention, one of pA₁ and pA₂ is a poly-alanine domain and the other of pA₁ and pA₂ is a poly-alanine domain having from two up to four, preferably, three alanine residues replaced by a respective amino acid residue selected from the group consisting of isoleucine and valine. Hence, in this embodiment, the recombinant spider silk protein has one poly-alanine domain preferably consisting of alanine residues and one poly-alanine domain that comprises two up to four, preferably, three amino acid residues individually selected among isoleucine and valine, in addition to alanine residues. In this embodiment, the isoleucine and/or valine residues do not necessarily have to be every third or fourth residue in the other of pA₁ and pA₂.

Another aspect of the invention relates to a silk fiber made of a recombinant spider silk protein according to the invention. Hence, this aspect relates to silk fiber, sometimes referred to as silk polymer, comprising a recombinant spider silk protein according to the invention. The silk fiber is then obtained by spinning a so called spinning dope comprising the recombinant spider silk protein according to the invention into the silk fiber, which is further described herein.

Strength, strain at break, toughness modulus, diameter and other mechanical properties as referred to herein relate to average values of the mechanical properties as determined when testing a plurality of silk fibers.

In an embodiment, the silk fibers of the invention have an average strength of at least 50 MPa, preferably at least 60 MPa and more preferably at least 70 MPa, such as at least 80 MPa.

In an embodiment, the silk fibers of the invention have an average strain at break of at least 60%, preferably at least 70%, and more preferably at least 100%, such as at least 125%.

In an embodiment, the silk fibers of the invention have an average toughness modulus of at least 25 MJ/m³, preferably at least 35 MJ/m³, and more preferably at least 45 MJ/m³, such as at least 75 MJ/m³.

The silk fiber could have an average diameter of from one or a few μm up to several tens of μm. For instance, the average diameter of the silk fiber is from 1 μm up to 100 μm, preferably from 5 μm up to 50 μm and more preferably from 7.5 up to 15 μm.

The present invention also relates to a synthetic material comprising a silk fiber according to the invention.

Illustrative examples of a synthetic material comprising, or made of, silk fibers of the invention include textile materials, such as filaments, yarns, ropes, and woven material. Such textile materials may benefit from the high tensile strength of the silk fiber. Other examples of synthetic materials include pliant energy absorbing materials, such as armor and bumpers. The silk fibers of the invention can also be used in medical applications, such as in sutures, compression bandages, etc. Additionally the silk fibers can be used in scaffolds and material in tissue engineering, implants and other cell scaffold-based materials.

The present invention also relates to a nucleic acid molecule encoding a recombinant spider silk protein according to the invention.

Nucleic acid molecule as used herein includes polynucleotide, oligonucleotide, and nucleic acid sequence, and generally means a polymer of DNA or RNA, which may be single-stranded or double-stranded, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. Nucleic acid molecule also includes complementary DNA (cDNA) and messenger RNA (mRNA).

Illustrative, but non-limiting, examples of such nucleic acid molecules are presented in SEQ ID NO: 68 for the recombinant spider silk protein in SEQ ID NO: 39, in SEQ ID NO: 69 for the recombinant spider silk protein in SEQ ID NO: 40 and in SEQ ID NO: 70 for the recombinant spider silk protein in SEQ ID NO: 34.

In an embodiment, the nucleic acid molecule is an isolated nucleic acid molecule.

A further aspect of the invention relates to an expression vector comprising a nucleic acid molecule according to the invention.

The expression vector comprises at least one nucleic acid molecule comprising coding sequences that can be expressed, such as transcribed and translated, in a cell, often denoted host cell, comprising the expression vector. The expression vector is in an embodiment selected among DNA molecules, RNA molecules, plasmids, episomal plasmids and virus vectors.

The expression vector then comprises the nucleic acid molecule operatively coupled to a promoter to enable transcription thereof in a host cell. The promoter could be any promoter that is constitutively active or inducibly active in the host cell. An illustrative, but non-limiting, example of a promoter that could be used in Escherichia coli host cells is the T7 promoter. Protein production could then be induced in the host cell by addition of isopropyl β-D-1-thiogalactoside.

In an embodiment, the expression vector is an isolated expression vector.

Yet another aspect of the invention relates to a host cell comprising the expression vector according to the invention.

The nucleic acid molecule or expression vector can then be transcribed in the host cell to produce the recombinant spider silk protein in the host cell.

Various such host cells can be used according to the invention including, but not limited to, bacteria, yeast, mammalian cells, plant cells, and insect cells. It is currently preferred to produce the recombinant spider silk proteins of the invention in prokaryotic cells, preferably bacteria, such as E. coli.

The recombinant spider silk protein can then be produced by the host cell, for instance, by culturing the host cell according to the invention in conditions allowing the production of the recombinant spider silk protein and isolating the spider silk protein from the culture. In a particular embodiment, the spider silk protein is isolated from the cytosol of the host cells.

The present invention also relates to a method for producing a silk fiber. The method comprises extruding a spinning dope comprising the recombinant spider silk protein of the present invention into an aqueous buffer having an acidic pH to induce polymerization of the recombinant spider silk protein into a silk fiber. The method also comprises isolating the silk fiber from the aqueous buffer.

In an embodiment, the spinning dope comprises at least 100 mg/ml of the recombinant spider silk protein, preferably at least 150 mg/ml and more preferably at least 200 mg/ml of the recombinant spider silk protein.

In an embodiment, the aqueous buffer is an acetate buffer having a pH equal to or below 6, preferably equal to or below 5.5. In a particular embodiment, the aqueous buffer preferably also has a pH equal to or larger than 4, preferably equal to or larger than 4.5. In a preferred, embodiment, the aqueous buffer has a pH of about 5.

The recombinant spider silk proteins advantageously enable the production of silk fibers in industrially compatible set-ups using metal nozzles as disclosed in Example 2. Furthermore, the so-produced silk fibers can be drawn after spinning.

### EXAMPLES

### Example 1—Engineered Spider Silk Proteins for Biomimetic Spinning of Fibers

This Example involved usage of protein engineering to generate mini-spidroins produced at high yields in prokaryotic hosts and that were used to generate strong biomimetic artificial spider silk fibers. The Zipper database (Goldschmidt et al., Proceedings of the National Academy of Sciences of the United States of America 107:3487 (2010)) was used to screen a large panel of mini-spidroins with designed modifications of the poly-Ala blocks and candidates with low Rosetta energies were chosen for heterologous expression. Soluble target proteins were identified, characterized biochemically, and spun into fibers using a biomimetic spinning device. The mechanical performance of the fibers revealed that engineering of the repeat domain of mini-spidroins was possible and resulted in fibers with increased tensile strength.

### Results

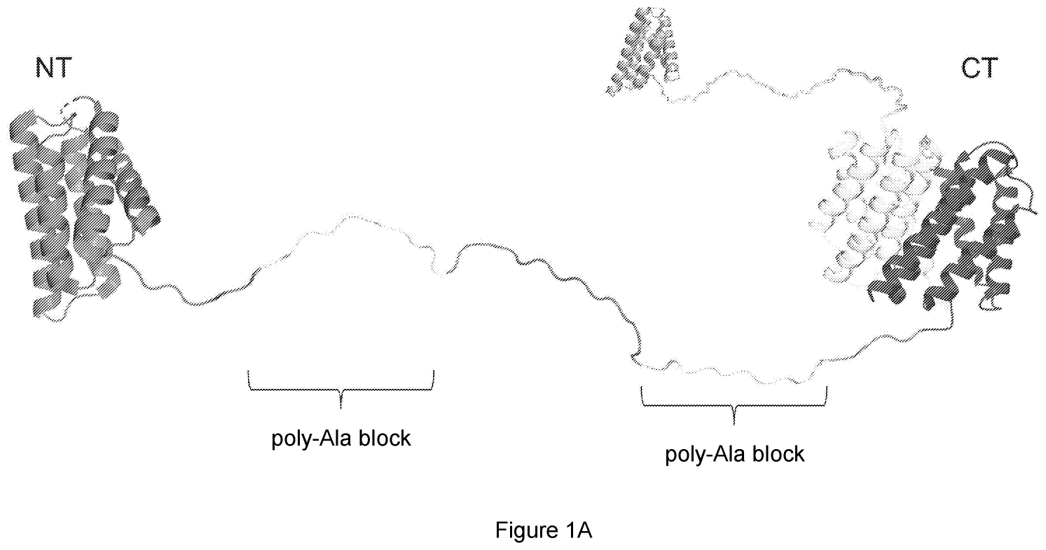

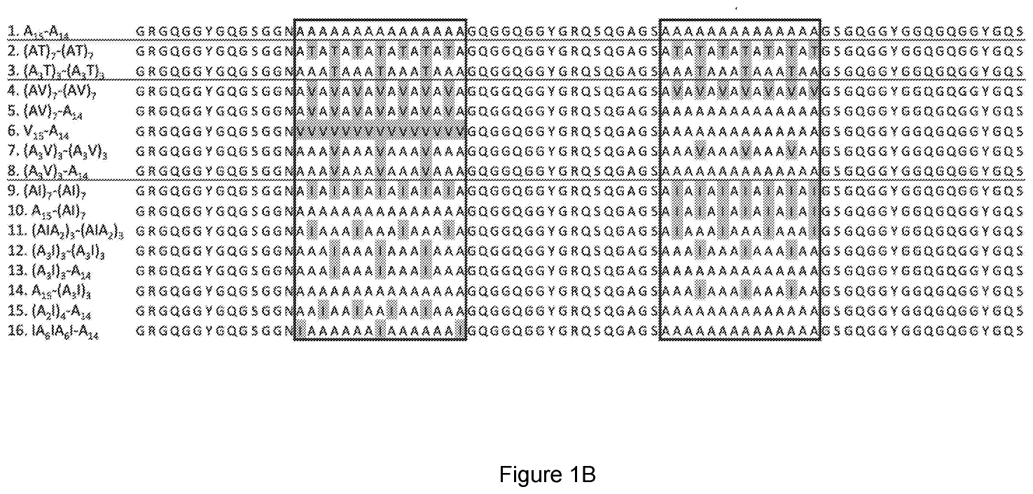

Based on the β-strand/cx-helix propensity ratios of amino acid residues as well as their hydrophobicity, Ile (I) and Val (V) were chosen to design 13 different constructs with substitutions in the poly-Ala blocks of the original NT2RepCT sequence (referred to as A₁₅-A₁₄ to reflect the composition of the two poly-Ala blocks), (FIG. 1). Additionally, the less hydrophobic residue Thr (T) was used since it is branched at the β-carbon and hence favors β-strand conformation (Koehl and Levitt, Proceedings of the National Academy of Sciences of the United States of America 96:12524 (1999); Chou and Fasman, Biochemistry 13:211 (1974)).

FIG. 1B shows the amino acid sequences of the repetitive regions from A₁₅-A₁₄ and engineered constructs with substitutions indicated. Substitutions were mainly introduced at every second position resulting in β-strands with mutated side chains on the same side. Mutations were introduced in either both, e.g., (AV)₇-(AV)₇ or only in one of the poly-Ala blocks, e.g., (AV)₇-A₁₄. The number of substitutions varied between 15, e.g., V₁₅-A₁₄, in which all Ala are replaced by Val in the first poly-Ala block, and 3, e.g. (A₃V)₃-A₁₄, which contains Val substitution at every fourth position in the first poly-Ala block. A few additional constructs were designed to analyze the impact of the position of the substituted residues, e.g., (A₃I)₃-A₁₄, A₁₅-(A₃I)₃ and IA₆IA₆I-A₁₄ that all have three Ile substitutions but in different locations.

The packing of β-sheets in amyloid-like fibrils involve steric zippers, which are also found in spider silk β-sheet crystals. Steric zippers are formed by tightly bound-strands with high complementarity of the involved side chains. The Zipper database predicts the stability and propensity of hexapeptides in a given amino acid sequence to form steric zippers by calculating the energies of the inter-strand interactions. Energies equal or below-23 kcal/mol suggest a high propensity to form steric zippers (Goldschmidt et al., Proceedings of the National Academy of Sciences of the United States of America 107:3487 (2010).

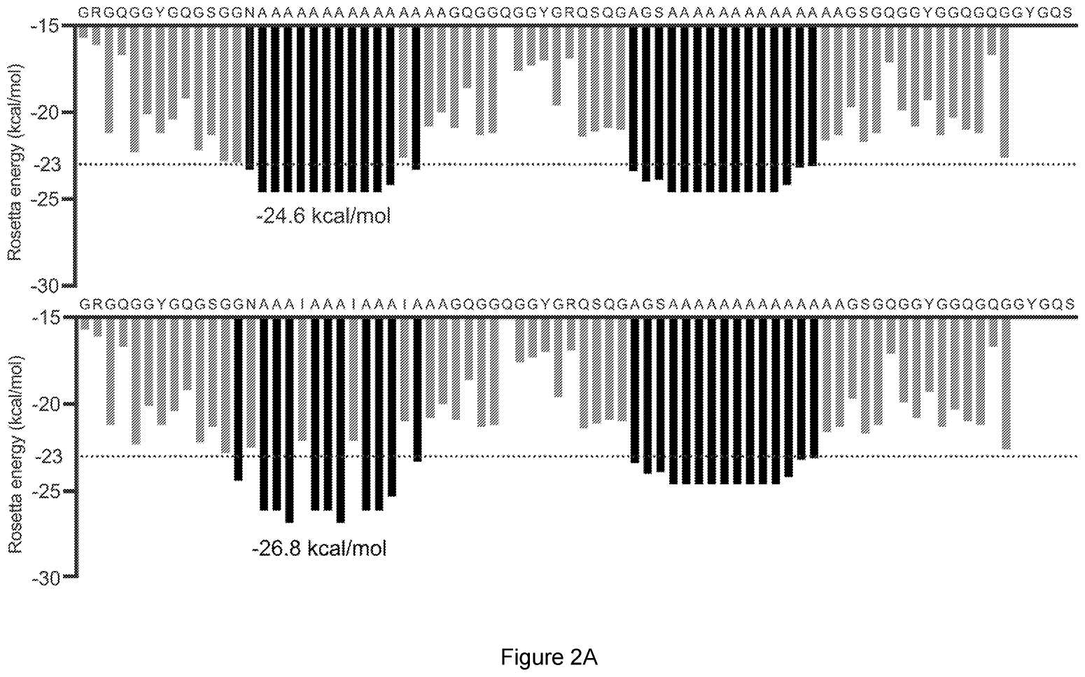

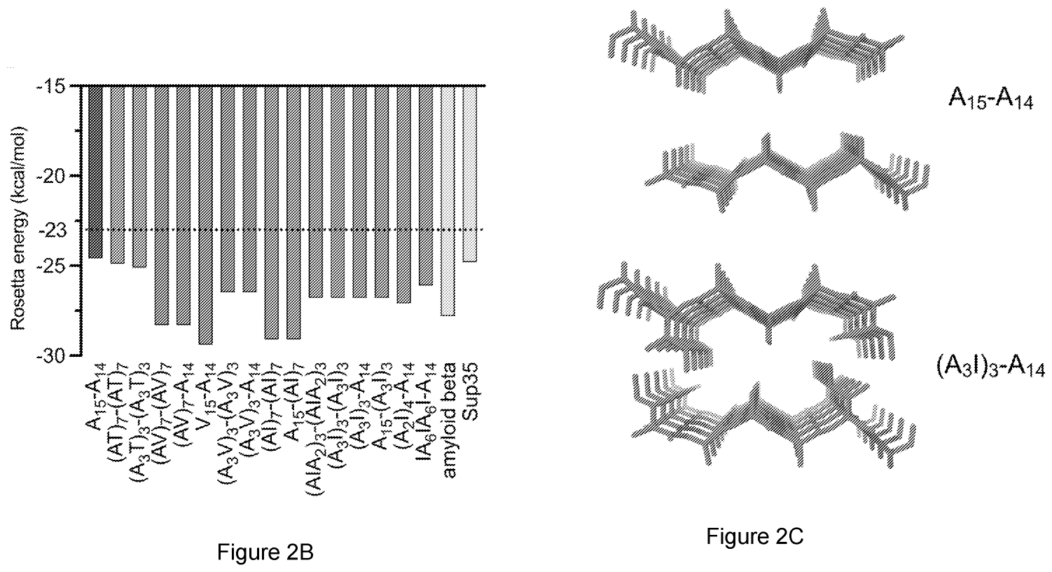

FIG. 2A shows the Rosetta energies estimated for constructs A₁₅-A₁₄ and (A₃I)₃-A₁₄ (corresponding profiles for all engineered mini-spidroins are summarized in Table 1 and FIG. 2B). The hexapeptides in the poly-Ala region of the A₁₅-A₁₄ construct have low Rosetta energies (−24.6 kcal/mol) and, thus, should be able to form steric zippers (FIG. 2C). All designed constructs contain at least one hexapeptide with a Rosetta energy lower than that of A₁₅-A₁₄ (Table 1), ranging from −24.9 to −29.4 kcal/mol. Generally, the effect on the Rosetta energies increased with an increasing number of hydrophobic replacements in the poly-Ala region.

TABLE 1
Hexapeptides with lowest Rosetta energies and
hydropathy of the engineered mini-spidroins.

| | Example | Rosetta energy | | |
|---|---|---|---|---|
| Construct | of hexapeptide | SEQ ID NO: | (kcal/mol) | Hydropathy |
| A₁₅-A₁₄ | AAAAAA | 17 | −24.6 | −0.168 |
| (AT)₇-(AT)₇ | ATATAT | 18 | −24.9 | −0.617 |
| (A₃T)₃-(A₃T)₃ | AAATAA | 19 | −25.1 | −0.36 |
| (AV)₇-(AV)₇ | AVAVAV | 20 | −28.3 | 0.263 |
| (AV)₇-A₁₄ | AVAVAV | 20 | −28.3 | 0.047 |
| V₁₅-A₁₄ | VVVVVV | 21 | −29.4 | 0.294 |
| (A₃V)₃-(A₃V)₃ | AVAAAV | 22 | −26.5 | 0.017 |
| (A₃V)₃-A₁₄ | AVAAAV | 22 | −26.5 | −0.076 |
| (AI)₇-(AI)₇ | AIAIAI | 23 | −29.1 | 0.317 |
| A₁₅-(AI)₇ | AIAIAI | 23 | −29.1 | 0.074 |
| (AIA₂)₃-(AIA₂)₃ | AIAAAI | 24 | −26.8 | 0.109 |
| (A₃I)₃-(A₃I)₃ | AIAAAI | 24 | −26.8 | 0.04 |
| (A₃I)₃-A₁₄ | AIAAAI | 24 | −26.8 | −0.064 |
| A₁₅-(A₃I)₃ | AIAAAI | 24 | −26.8 | −0.064 |
| (A₂I)₄-A₁₄ | AAIAAI | 25 | −27.1 | −0.029 |
| IA₆IA₆I-A₁₄ | AAAIAA | 26 | −26.1 | −0.064 |

Of the 15 designed proteins, seven were overexpressed and six were highly overexpressed in Escherichia coli BL21 cells (Table 2). Constructs with Val substitutions had lower expression levels than corresponding constructs with Ile substitutions, but the number of substitution and the hydrophobicity did not have any general impact on expression levels. The (AT)₇-(AT)₇ construct did not express well, which could be due to that this repeat was designed to resemble a “CAT tail”, which is known to lead to aggregation of the nascent polypeptide chain and to degradation by the proteasome (Shen et al., Science 347:75 (2015)).

In addition to A₁₅-A₁₄, seven of the constructs were found mainly in the soluble fraction after cell lysis in 20 mM Tris-HCl, and four constructs were in both the soluble and insoluble fraction (Table 2). Increased hydrophobicity, number of substitutions and lower Rosetta energies correlated with lower solubility after cell lysis. Nine of the 15 designed constructs plus the control A₁₅-A₁₄ yielded sufficient soluble protein for purification. Non-denaturing immobilized metal affinity chromatography yielded between 4 to 243 mg of pure target protein per 1 L shake flask culture (average of 10×1 L cultures). Notably, six of the engineered mini-spidroins gave very high yields (>100 mg/L Table 2). (AV)₇-(AV)₇, (AV)₇-A₁₄ and V₁₅-A₁₄ expressed well but were insoluble after lysis, likely due to high hydrophobicity of the engineered segments. Expression and purification of the A₁₅-(AI)₇ and (AIA₂)₃-(AIA₂)₃ constructs did not result in enough soluble protein for further characterization. The constructs that showed intermediate to high expression levels but were insoluble after cell lysis were treated with 8 M urea but could not be solubilized to the extent needed for enabling purification of enough protein for fiber spinning (not shown).

The position of the Ile replacements within one Ala block had an impact on the protein yield but whether these were located in the first or second poly-Ala block did not matter. For example, (A₃I)₃-A₁₄ and A₁₅-(A₃I)₃ both have three Ile substitutions in the first and second poly-Ala block, respectively, and showed comparable yields. In contrast, (A₃I)₃-A₁₄ and IA₆IA₆I-A₁₄ have the same number of Ile replacements in the first block, but their location differed as did the yield (207 vs 139 mg/L culture for (A₃I)₃-A₁₄ and IA₆IA₆I-A₁₄, respectively).

TABLE 2
Summary of number of substitutions, expression levels, solubility after cell
lysis, protein yield and spinnability into fibers of the engineered proteins.

| | | | Solubility | Average | |
|---|---|---|---|---|---|
| | Number of | Expression | after | protein yield | Spinnability |
| Construct | substitutions | levels | cell lysis | (mg/L culture) | into fibers |
| 1. A₁₅-A₁₄ | 0 | +++ | +++ | 250 | +++ |
| 2. (AT)₇-(AT)₇ | 14 | + | − | − | − |
| 3. (A₃T)₃-(A₃T)₃ | 6 | ++ | +++ | 58* | +++ |
| 4. (AV)₇-(AV)₇ | 14 | +++ | 0 | − | − |
| 5. (AV)₇-A₁₄ | 7 | +++ | 0 | − | − |
| 6. V₁₅-A₁₄ | 15 | +/++¹ | 0 | − | − |
| 7. (A₃V)₃-(A₃V)₃ | 6 | +++ | +++ | 139* | +++ |
| 8. (A₃V)₃-A₁₄ | 3 | ++ | +++ | 216 | +++ |
| 9. (Al)₇—(Al)₇ | 14 | + | + | 4* | − |
| 10. A₁₅-(Al)₇ | 7 | + | + | − | − |
| 11. (AlA₂)₃-(AlA₂)₃ | 8 | ++ | + | − | − |
| 12. (A₃I)₃-(A₃I)₃ | 6 | +++ | +++ | 94* | + |
| 13. (A₃I)₃-A₁₄ | 3 | +++ | +++ | 207 | +++ |
| 14. A₁₅-(A₃I)₃ | 3 | +++ | +++ | 233 | +++ |
| 15. (A₂I)₄-A₁₄ | 4 | ++ | +++ | 243 | +++ |
| 16. IA₆IA₆I-A₁₄ | 3 | ++ | ++ | 139 | − |

Expression levels, solubility after cell lysis and spinnability into fibers are rated from very high (+++), intermediate (++), low (+) and not at all (0).
Rating of expression level and solubility after cell lysis were estimated by appearance of the target band on SDS-PAGE.
(−) indicate not tested.
¹indicates degradation during expression.
*marks purification using gravity columns instead of FPLC.

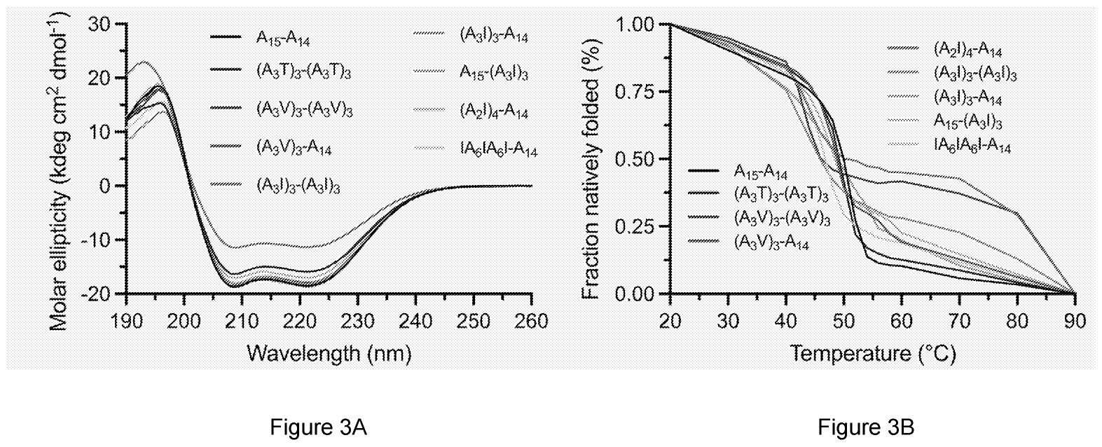

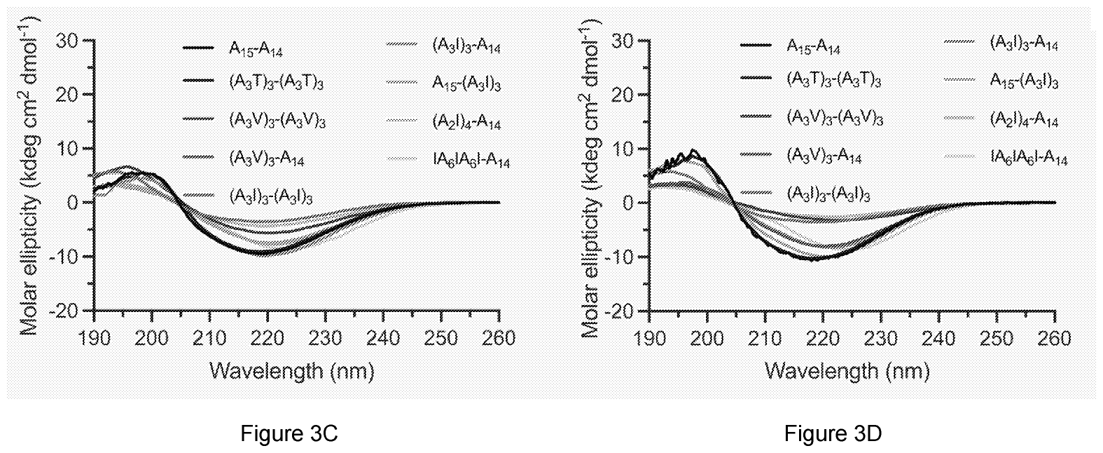

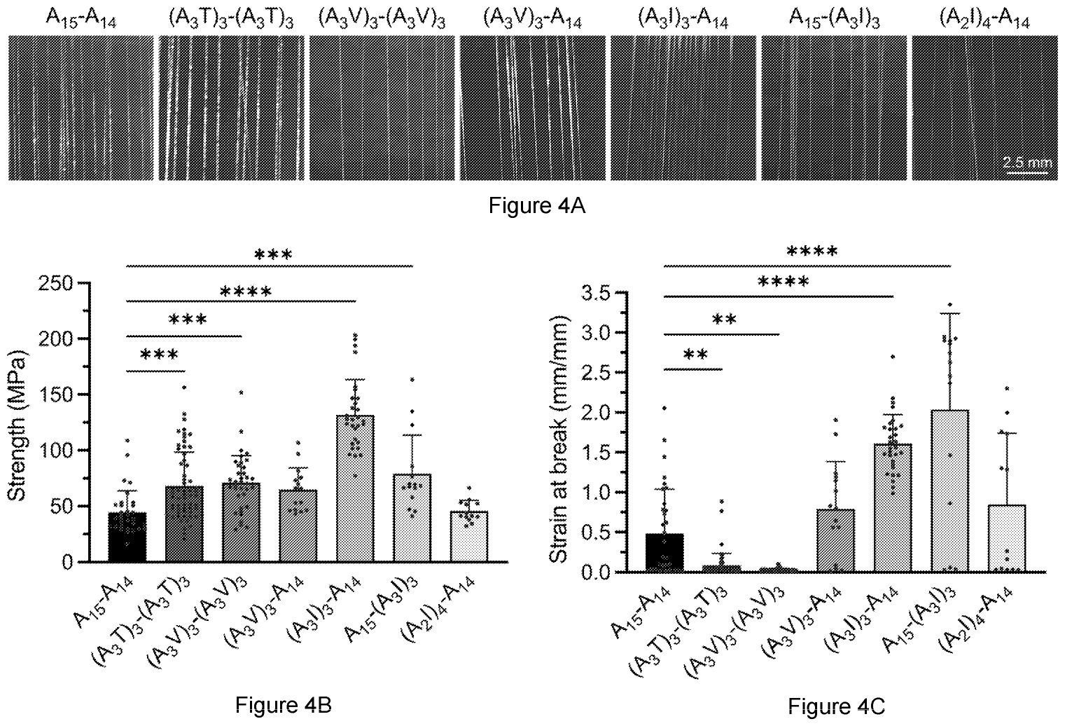

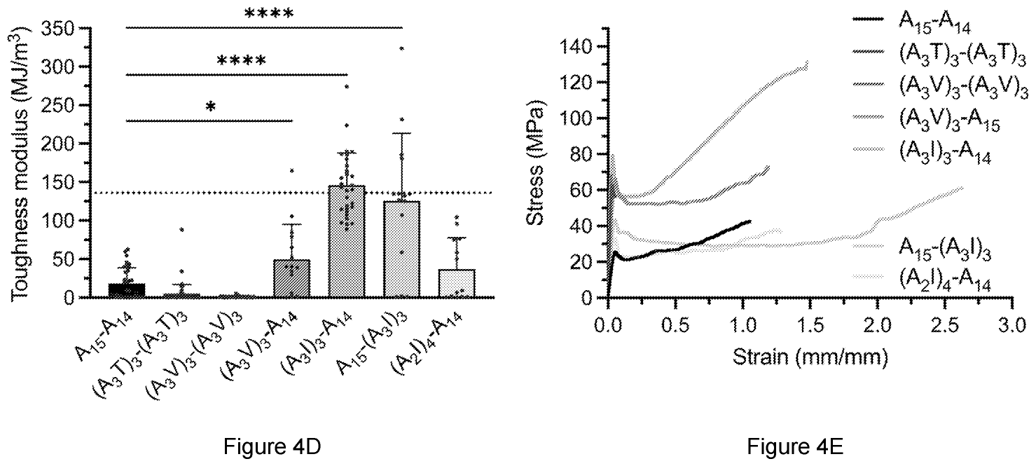

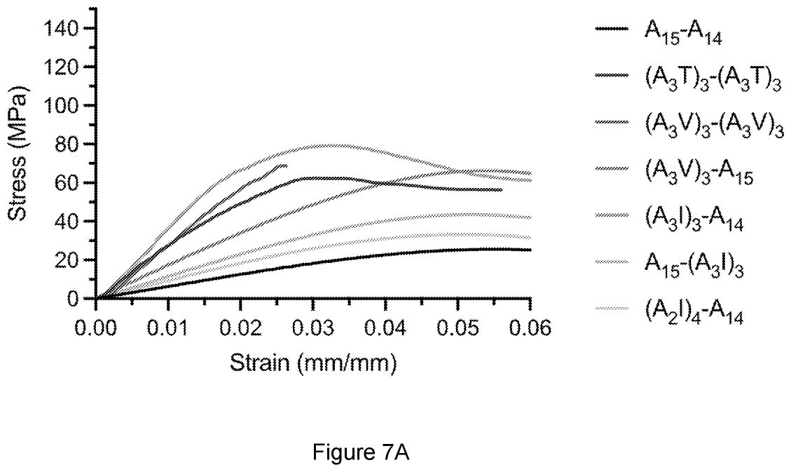

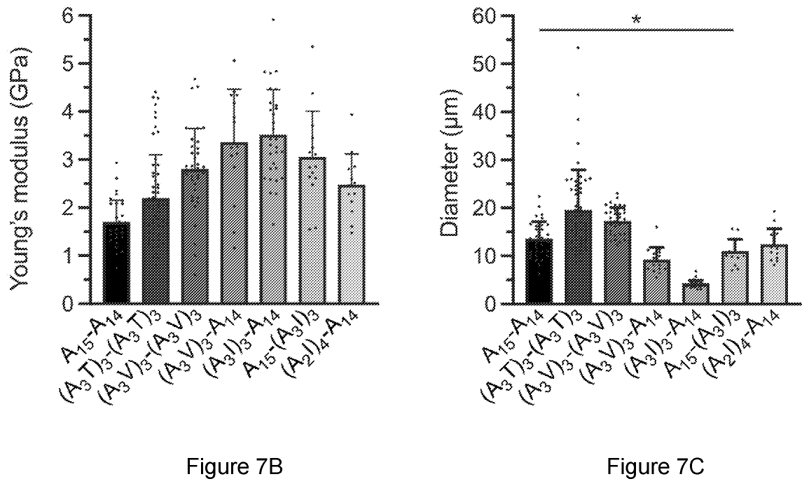

Next, we investigated the secondary structure content and the thermal stability of the purified constructs by circular dichroism (CD) spectroscopy (FIG. 3). We found that all constructs had an overall cx-helical secondary structure (FIG. 3A), which indicates that the amino acid substitutions did not affect the secondary structure of the soluble proteins to any large extent. Heating to 90° C. led to a decreased signal for all constructs and concomitant transition to β-sheet dominated secondary structures (FIG. 3C). The heat-induced conformational changes were irreversible upon cooling of the samples (FIG. 3D). Melting curves for all constructs showed that the proteins unfolded around 46-50° C., which means that the substitutions only had a minor effect on the thermal stability of the proteins (FIG. 3B). Out of the nine engineered mini-spidroins that were successfully purified (excluding A₁₅-A₁₄), eight could be concentrated to at least 200 mg/ml to generate spinning dopes, while (AI)₇-(AI)₇ yielded too little protein (Table 2). The dopes made from the eight constructs were transferred to syringes and extruded through a thin glass capillary into a low pH aqueous buffer according to a previously described biomimetic spinning procedure (Greco et al., Molecules 25:3248 (2020); Andersson et al., Nature Chemical Biology 13:262 (2017). Seven engineered mini-spidroins could be spun into fibers, only the IA₆IA₅I-A₁₄ protein aggregated prematurely in the syringe. One of the mini-spidroins, (A₃I)₃-(A₃I)₃, formed fibers that were too fragile to be retrieved. The reason for the poor integrity of the (A₃I)₃-(A₃I)₃ fibers is not known but was not related to premature aggregation in the dope. The other six engineered fiber types, plus the A₁₅-A₁₄ fibers, were successfully collected onto a motorized wheel at the end of the spinning bath. There was no difference in the appearance of the spun fibers (FIG. 4A) and the diameter of the different fiber types, determined by light microscopy, varied between 4 and 19 μm (FIG. 7C, Table 3).

TABLE 3
Mechanical properties of spinnable constructs and their standard deviation.

| | | | Toughness | | Young's |
|---|---|---|---|---|---|
| | Strength | Strain at break | modulus | Diameter | modulus |
| | (MPa) | (%) | (MJ/m³) | (μm) | (MPa) |
| A₁₅-A₁₄ | 44.09 ± 19.64 | 47.96 ± 55.82 | 18.19 ± 20.34 | 13.40 ± 3.70 | 1685 ± 466 |
| (A₃T)₃-(A₃T)₃ | 67.80 ± 30.58 | 8.31 ± 15.15 | 4.70 ± 12.34 | 19.41 ± 8.51 | 2183 ± 921 |
| (A₃V)₃-(A₃V)₃ | 70.76 ± 24.37 | 3.26 ± 1.82 | 1.31 ± 1.07 | 17.12 ± 2.95 | 2786 ± 861 |
| (A₃V)₃-A₁₄ | 64.51 ± 19.73 | 78.59 ± 59.89 | 49.58 ± 45.26 | 9.09 ± 2.68 | 3348 ± 1121 |
| (A₃I)₃-A₁₄ | 131.63 ± 31.87 | 160.44 ± 37.00 | 145.63 ± 42.18 | 4.16 ± 0.78 | 3501 ± 948 |
| A₁₅-(A₃I)₃ | 78.78 ± 34.87 | 203.52 ± 120.39 | 125.33 ± 87.99 | 10.88 ± 2.59 | 3045 ± 964 |
| (A₂I)₄-A₁₄ | 45.41 ± 9.73 | 84.51 ± 89.26 | 37.01 ± 40.61 | 12.11 ± 3.33 | 2463 ± 653 |
| (A₃I)₃-A₁₄^{a} | 95.68 ± 39.53 | 150.76 ± 57.09 | 90.96 ± 45.19 | 6.13 ± 4.00 | 2854 ± 868 |

^{a}protein expressed in a bioreactor

The tensile strength of all fibers spun from engineered proteins increased significantly compared to A₁₅-A₁₄ except for (A₃V)₃-A₁₄ and (A₂I)₄-A₁₄ (FIG. 4B and Table 3). The two similar fiber types (A₃I)₃-A₁₄ and A₁₅-(A₃I)₃ displayed the highest increase in strength, the former reaching 131 MPa, which is almost three times higher than that of A₁₅-A₁₄ (FIG. 4B). This indicates that rational protein engineering of the spidroin poly-Ala blocks indeed can result in increased fiber tensile strength. Unexpectedly, the introduced amino acid substitutions also had a high impact on the extensibility of the fiber as the strain at break varied from 3.3 to 203.5% (FIG. 4C and Table 3).

The two strongest fiber types ((A₃I)₃-A₁₄ and A₁₅-(A₃I)₃) displayed an exceptional increase in strain (160 and 204%, respectively), while (A₃V)₃-A₁₄, (A₂I)₄-A₁₄ fibers showed moderately increased strain (79 and 85%, respectively) compared to A₁₅-A₁₄ (45%). (A₃T)₃-(A₃T)₃ and (A₃V)₃-(A₃V)₃ fibers were the least extensible (3.3 and 8.3%, respectively). Apparently, the mechanical properties of artificial spider silk fibers can be significantly improved by introducing Ile in every fourth position in the first or second poly-Ala block. These two mini-spidroins, (A₃I)₃-A₁₄ and A₁₅-(A₃I)₃, formed fibers with a toughness modulus that is comparable to native dragline silk (146 and 125 MJ/m³, respectively, versus 136 MJ/m³ for a native dragline silk from Argiope argentata, Blackledge and Hayashi, Journal of Experimental Biology 209:2452 (2006), (FIG. 4D). Fibers formed by (A₃V)₃-A₁₄ and (A₂I)₄-A₁₄ also reached a significantly higher toughness modulus than A₁₅-A₁₄ (50 and 37 MJ/m³, respectively, versus 18 MJ/m³).

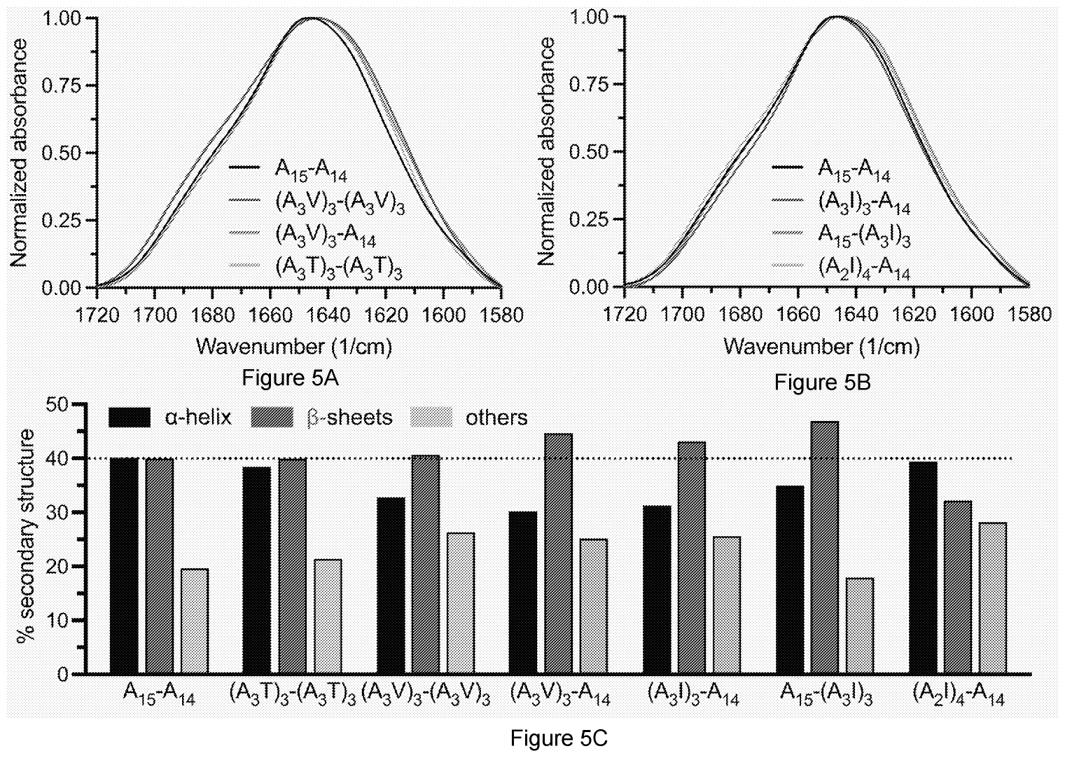

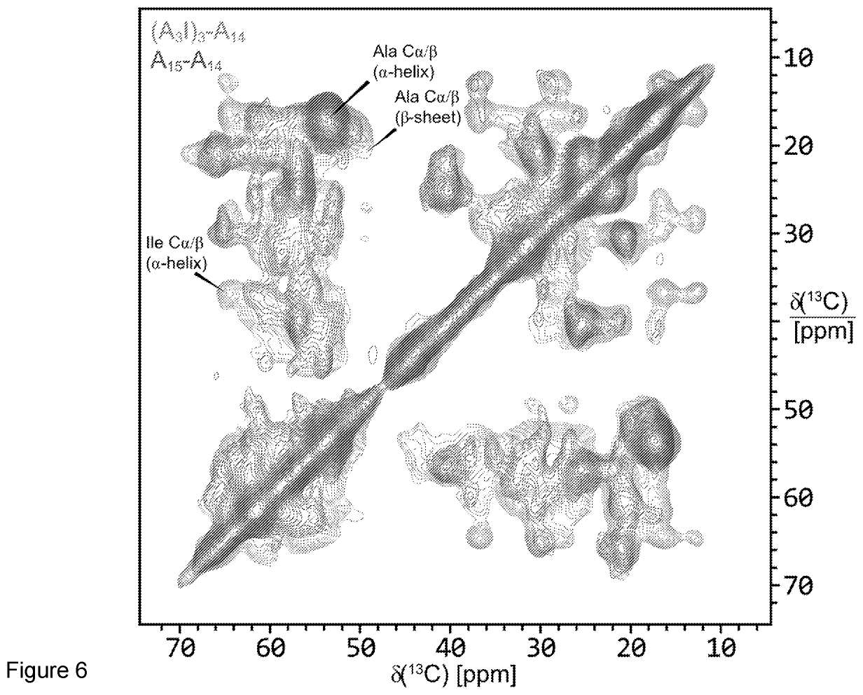

To investigate the link between fiber secondary structure content and mechanical properties, we used Attenuated Total Reflection Fourier-transform infrared (ATR-FTIR) spectroscopy. The results, shown in FIG. 5, indicated that no large differences in secondary structure content between fibers were detected, but (A₃V)₃-A₁₄, (A₃I)₃-A₁₄ and A₁₅-(A₃I)₃ had a slightly increased β-sheet content, along with decreased α-helix/random coil content compared to A₁₅-A₁₄ fibers. However, the (A₃V)₃-(A₃V)₃ and (A₂l)₄-A₁₄ fibers failed to show increased β-sheet content compared to A₁₅-A₁₄ fibers and we could detect no strong correlations between secondary structure content and mechanical properties of the fiber. Thus, ATR-FTIR spectroscopy of the different fiber types did not detect any significant differences in secondary structure content. Therefore, we decided also to use solid-state NMR spectroscopy to investigate the unmodified fibers (A₁₅-A₁₄) and the best performing engineered fibers, (A₃I)₃-A₁₄. More Ala residues were found in a β-sheet conformation in (A₃I)₃-A₁₄ compared to A₁₅-A₁₄ fibers (FIG. 6).

The altered mechanical properties of the fibers made from the engineered spidroins indicate that intermolecular interactions in the spidroins are affected. In the native dragline silk fiber, pulling the fiber first results in reversible deformation of the amorphous regions up until the yielding point, after which the hydrogen bonds in the amorphous region break, resulting in softening of the material. When the amorphous protein chains are extended, the load is transferred onto the β-sheet crystals leading to a stiffening of the fiber. Upon further increased load, the β-sheet crystals undergo stick-slip deformation and the fiber breaks. The increased tensile strength of the fibers made from engineered proteins suggests that our strategy to increase the β-strand propensity and inter-β-sheet interactions indeed can result in stronger fibers, although some of the engineered fibers concomitantly displayed a decreased strain. Theoretically, increased β-sheet formation and intermolecular interactions in the stacked β-sheets could not only result in increased fiber strength, but also increased extensibility, since the amorphous region would be allowed to extend fully before the load is transferred to the crystalline region. In lack of poly-Ala β-sheet crystals, as in the A₁₅-A₁₄ fibers, the intermolecular contacts may be too weak to allow a full extension of the amorphous protein chains before fiber failure. At the same time, it may be disadvantageous that all β-sheets stack in crystals since only about 40% of the Ala residues in the native dragline silk are found in this conformation and the rest form less ordered β-sheets. In this study, introducing replacements in both poly-Ala blocks resulted in fibers with dramatically reduced strain, which suggests a suboptimal packing of the proteins in the fiber.

Since the (A₃I)₃-A₁₄ fibers displayed superior mechanical properties, these fibers are attractive candidates for bulk-scale production. Previously, A₁₅-A₁₄ has been shown to express at very high levels (˜21 g/L) in a bioreactor-based E. coli fed-batch culture (Schmuck et al., Materials Today 50:16 (2021)). Following the same protocol, the expression level of (A₃I)₃-A₁₄ amounted to 13 g/L and the final yield after purification using an automated purification protocol was 8.9 g/L. To our knowledge, these yields are the second highest reported for any recombinant spidroin produced in E. coli and line with what is required for economically viable bulk production. After purification, (A₃I)₃-A₁₄ was concentrated to 300 mg/ml and could easily be spun into fibers. Notably, 8.9 g recombinant silk protein is enough to produce an approximately 18 km long fiber.

Using biological principles, we employed protein engineering to design mini-spidroins with predicted increased β-sheet propensities and increased inter-β-sheet binding strengths. Prokaryotic expression, protein purification and biomimetic fiber spinning resulted in four different types of fibers with significantly improved tensile strength compared to the original mini-spidroin. Using this strategy, we successfully produced the first biomimetic fibers with toughness values matching those of native dragline silk fibers. Finally, we show that these fibers can be produced at very high yields in bioreactors, vouching for feasible large-scale production.

### Experimental Section

### Designed Mini-Spidroins

All expressed proteins were composed of a 6×His-tag (MGHHHHHH, SEQ ID NO: 71), an NT from Euprosthenops australis MaSp1 (SEQ ID NO: 53) and a CT from Araneus ventricosus minor ampullate spidroin (MiSp) (SEQ ID NO: 54). Between NT and CT, a repetitive part was inserted containing two poly-Ala and three glycine-rich repeats from E. australis MaSp1 (NT2RepCT) as described previously (Andersson et al., Nature Chemical Biology 13:262 (2017)). Engineered variants were designed that contained amino acid residue substitutions in the poly-Ala blocks of the repetitive region as described in the results section. Note that the constructs were named after their substitutions in the poly-Ala blocks but contained NT, CT and the glycine-rich regions as well, e.g., NT2RepCT is referred to as A₁₅-A₁₄.

Amino acid sequences corresponding to the designed repeat regions were converted into gene sequences and codon optimized for expression in E. coli (Geneious), ordered from Eurofins Genomics, Germany, and subcloned between NT and CT (using EcoRI and BamHI restriction sites) of the existing NT2RepCT plasmid (Andersson et al., Nature Chemical Biology 13:262 (2017).

The full sequences below have the general layout His-NT-Linker1-pG₁-pA₁-pG₂-pA₂-pG₃-Linker2-CT.

A₁₅-A₁₄,
SEQ ID NO: 27
MGHHHHHHMSHTTPWTNPGLAENEMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAAAAAAAAAAAAAAGQGGQGGYGRQS
QGAGSAAAAAAAAAAAAAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(AT)₇-(AT)₇,
SEQ ID NO: 28
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNATATATATATATATAGQGGQGGYGRQS
QGAGSATATATATATATATGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(A₃T)₃-(A₃T)₃,
SEQ ID NO: 29
MGHHHHHHMSHTTPWTNPGLAENEMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAATAAATAAATAAAGQGGQGGYGRQS
QGAGSAAATAAATAAATAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(AV)₇-(AV)₇,
SEQ ID NO: 30
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAVAVAVAVAVAVAVAGQGGQGGYGRQS
QGAGSAVAVAVAVAVAVAVGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(AV)₇-A₁₄,
SEQ ID NO: 31
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAVAVAVAVAVAVAVAGQGGQGGYGRQS
QGAGSAAAAAAAAAAAAAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
V₁₅-A₁₄,
SEQ ID NO: 32
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNVVVVVVVVVVVVVVVGQGGQGGYGRQS
QGAGSAAAAAAAAAAAAAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(A₃V)₃-(A₃V)₃,
SEQ ID NO: 33
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAAVAAAVAAAVAAAGQGGQGGYGRQS
QGAGSAAAVAAAVAAAVAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(A₃V)₃-A₁₄,
SEQ ID NO: 34
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAAVAAAVAAAVAAAGQGGQGGYGRQS
QGAGSAAAAAAAAAAAAAAGSGQGGYGGQGQGGYGQSGNSSVTSGGYGYGTSAAAGAGVAAG
SYAGAVNRLSSAEAASRVSSNIAAIASGGASALPSVISNIYSGVVASGVSSNEALIQALLEL
LSALVHVLSSASIGNVSSVGVDSTLNVVQDSVGQYVG
(AI)₇-(AI)₇,
SEQ ID NO: 35
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAIAIAIAIAIAIAIAGQGGQGGYGRQS
QGAGSAIAIAIAIAIAIAIGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
A₁₅-(AI)₇,
SEQ ID NO: 36
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAAAAAAAAAAAAAAGQGGQGGYGRQS
QGAGSAIAIAIAIAIAIAIGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(AIA₂)₃-(AIA₂)₃,
SEQ ID NO: 37
MGHHHHHHMSHTTPWTNPGLAENEMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAIAAAIAAAIAAAIAGQGGQGGYGRQS
QGAGSAIAAAIAAAIAAAIGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(A₃I)₃-(A₃I)₃,
SEQ ID NO: 38
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAAIAAAIAAAIAAAGQGGQGGYGRQS
QGAGSAAAIAAAIAAAIAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(A₃I)₃-A₁₄,
SEQ ID NO: 39
MGHHHHHHMSHTTPWTNPGLAENFMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAAIAAAIAAAIAAAGQGGQGGYGRQS
QGAGSAAAAAAAAAAAAAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
A₁₅-(A₃I)₃,
SEQ ID NO: 40
MGHHHHHHMSHTTPWTNPGLAENEMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAAAAAAAAAAAAAAGQGGQGGYGRQS
QGAGSAAAIAAAIAAAIAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
(A₂I)₄-A₁₄,
SEQ ID NO: 41
MGHHHHHHMSHTTPWTNPGLAENEMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNAAIAAIAAIAAIAAAGQGGQGGYGRQS
QGAGSAAAAAAAAAAAAAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG
IA₆IA₆I-A₁₄,
SEQ ID NO: 42
MGHHHHHHMSHTTPWTNPGLAENEMNSFMQGLSSMPGFTASQLDDMSTIAQSMVQSIQSLAA
QGRTSPNKLQALNMAFASSMAEIAASEEGGGSLSTKTSSIASAMSNAFLQTTGVVNQPFINE
ITQLVSMFAQAGMNDVSAGNSGRGQGGYGQGSGGNIAAAAAAIAAAAAAIGQGGQGGYGRQS
QGAGSAAAAAAAAAAAAAAGSGQGGYGGQGQGGYGQSGNSVTSGGYGYGTSAAAGAGVAAGS
YAGAVNRLSSAEAASRVSSNIAAIASGGASALPSVISNIYSGVVASGVSSNEALIQALLELL
SALVHVLSSASIGNVSSVGVDSTLNVVQDSVGQYVG

### Fibrillation Propensity and Hydrophobicity

The Zipper database (Goldschmidt et al., Proceedings of the National Academy of Sciences of the United States of America 107:3487 (2010)) was used to estimate the fibrillation propensity and Rosetta energies of engineered constructs (only the repetitive region) as silk has been proposed to form β-sheets that pack into crystals. The Zipper database calculates the Rosetta energy (Kuhlman and Baker, Proceedings of the National Academy of Sciences of the United States of America 97:10383 (2000)) and evaluate self-complementary binding of moving hexapeptides (Nelson et al., Nature 435:773 (2005); Sawaya et al., Nature 447:453 (2007). The Rosetta energy combines several free energy functions to model and analyze given protein structures, and energies equal or below-23 kcal/mol indicate high fibrillation propensity (Goldschmidt et al., Proceedings of the National Academy of Sciences of the United States of America 107:3487 (2010)). Lower energies imply higher stability of two β-strands in a zipper conformation. The hydrophobicity was calculated with https://web.expasy.org/protparam/(Wilkins et al, In 2-D Proteome Analysis Protocols, Humana Press, New Jersey, pp. 531-552 (1967); Gasteiger et al., The Proteomics Protocols Handbook, 571 (2005); Kyte and Doolittle, Journal of Molecular Biology 157:105 (1982)).

### Protein Expression Using Shake Flask Cultures

Protein expression was performed as described previously (Greco et al., Molecules 25:3248 (2020). In brief, the constructs were transformed in BL21 (DE3) E. coli cells and grown in Luria broth (Miller, VWR, USA) in shake flasks at 30° C. and 110 rpm containing kanamycin until the OD₆₀₀ reached 0.9. To induce recombinant protein expression, 0.15 mM isopropyl β-d-1-thiogalactopyranoside (final concentration; VWR, USA) was added and the temperature was lowered to 20° C. Expression took place overnight after which the cells were harvested and stored at −20° C.

### Protein Purification and Concentration

Cell lysis was done with a high-pressure cell disrupter (T-S Series Machine, Constant Systems Limited). Following centrifugation, the supernatant was purified by Ni-immobilized metal affinity column (IMAC), (Äkta start, GE Healthcare, USA or manual). After loading the supernatant on a HisPrep™ FF 16/10 or manual packed column (GE Healthcare, USA), the column was washed with 4-5 column volumes (CV) of 20 mM Tris-HCl followed by 4-5 CV of 2 mM imidazole in 20 mM Tris-HCl, pH 8. The protein was eluted with 200 mM imidazole in 20 mM Tris-HCl. After dialysis against 20 mM Tris-HCl, pH 8, the protein was analyzed by SDS-PAGE for quality control. Depending on the solubility of the construct, the proteins were concentrated to 200-400 mg/ml with centrifugal concentrators (Vivaspin 20, 10 kDa MWCO, GE Healthcare, USA) and then frozen at −20° C. until further use.

### CD Spectroscopy

Protein concentrations of 10 UM in 20 mM phosphate buffer were measured in a 300 μl cuvette with a 1 mm path length using a J-1500 CD spectrometer (JASCO, USA). Temperature scans were performed between 20 to 90° C. at a heating rate of 1° C. min-1 and spectra were recorded from 260 to 190 nm. After heating, the samples were cooled to 20° C. for 15 min to observe reversibility of the conformational changes. The means of five scans per temperature were smoothed and converted to molar residue ellipticity. Thermal unfolding curves were plotted by taking the molar residual ellipticity at 222 nm and the fraction natively folded was converted with the formula (CD_{measured}−CD_{end})/(CDₛₜₐᵣₜ−CD_{end}) and then normalized.

### Biomimetic Fiber Spinning

Artificial fiber spinning was performed similarly as described previously (Greco et al., Molecules 25:3248 (2020)). Round-glass capillaries (G1, Narishige, UK, inner diameter of 0.6 mm) were pulled with a Micro Electrode Puller (Stoelting co. 51217) to a diameter between 25 and 78 μm. A 1 ml syringe with Luer Lok tip (BD, USA) was filled with the concentrated proteins and connected to a 27 G steel needle (Braun, Germany). The needle was connected to the pulled-glass capillary via polyethylene tubing. The protein was ejected at a flow rate of 17 μl/min (neMESYS low-pressure syringe pump, Cetoni, Germany) into an 80 cm long bath containing spinning buffer (750 mM acetate buffer, 150 mM NaCl, pH 5.0) and rolled onto collection frames in air with minimal stretching of the fibers. Each construct was spun at least twice at different occasions.

### Mechanical Testing of the Fibers

Fibers were mounted with tape on paper frames with a square window (1 cm×1 cm) and the diameter of the fibers was measured with an optical microscope (Nikon, Japan) at 10 locations along each fiber and the average diameter was calculated. The frames were placed into a tensile tester (5943-Instron, USA equipped with a 5N load cell), cut and the fiber was pulled at a strain-rate of 6 mm/min. All the tests were performed at relative humidity lower than 35% to not affect the mechanical properties of the silk. The number of and types of fibers tested were: A₁₅-A₁₄ n=33, (A₃I)₃-A₁₄ n=30, (A₃T)₃-(A₃T)₃ n=60, (A₃V)₃-(A₃V)₃ n=38, (A₃V)₃-A₁₄ n=15, A₁₅-(A₃I)₃ n=13, (A₂I)₄-A₁₄ n=15. The engineering strength was calculated by dividing the measured force by the area of the cross-section (calculated from the apparent/maximal diameter assuming a circular cross-section). The engineering strain was calculated by dividing the displacement by the gauge length. Toughness modulus was obtained by calculating the area under the stress-strain curve and the Young's modulus was obtained from the slope at the initial linear elastic phase of the stress-strain curve.

### FTIR Spectroscopy

FTIR spectra of fiber bundles were recorded on a Vertex 70 instrument equipped with a diamond ATR unit (Platinum-ATR, Bruker, Germany) and a mercury cadmium telluride-detector (Bruker, Germany). The instrument was continuously purged with dried air and the spectra confirmed that water vapor correction was not necessary. 1000 scans with a resolution of 2 cm⁻¹ were recorded. Before every sample spectrum measurement, a background spectrum without a sample was recorded and used to calculate the absorbance spectrum. For each sample, 6 spectra were taken by pressing fiber bundles on the ATR crystal with 3 fiber bundles oriented perpendicular to the beam and 3 fiber bundles parallel to it.

The “Kinetics” software, written by Erik Goormaghtigh (Université Libre de Bruxelles, Belgium) was used to process the spectra. The 6 spectra of each sample were averaged and the baseline was subtracted (polynomial baseline with baseline points: 1740, 1730, 1580, and 1578 cm⁻¹) from the amide I band (1705-1595 cm⁻¹). The second derivative was calculated from the absorbance spectrum, smoothed with a 15-point Savitzky-Golay algorithm and scaled to match the absorbance values (factor=600). The absorbance and second derivative spectra were co-fitted simultaneously to analyze the secondary structure content (Baldassarre et al., Molecules 20, 12599 (2015)). Eight component bands were fitted (initial peak positions: 1695, 1680, 1669, 1651, 1633 1622, 1613, 1599 cm⁻¹) and bands were allowed to move ±5 cm⁻¹ from that initial center peak position. Each component band was assigned to a secondary structure according to literature (Goormaghtigh et al., Sub-cellular biochemistry 23:363 (1994); Jackson and Mantsch, Critical Reviews in Biochemistry and Molecular Biology 30:95 (1995); Venyaminov and Kalnin, Biopolymers 30:1259 (1990); Barth, Biochimica et Biophysica Acta—Bioenergetics 1767:1073 (2007); Barth and Zscherp, Quarterly Reviews of Biophysics 35:369 (2002). The component band fitted at a center peak position of ˜1695 cm⁻¹ was assigned to antiparallel β-sheets. The component band fitted at ˜1651 cm⁻¹ was assigned to α-helix/random structures. Bands at ˜1633, ˜1622, and ˜1613 cm⁻¹ were assigned to different types of β-sheets according to a study of Bombyx mori silk fibers (Carissimi et al., Polymers 12:1 (2020): the 1633 cm⁻¹ band likely corresponds to distorted or twisted β-sheets, while the ˜1622 and ˜ 1613 cm⁻¹ bands were assigned to more planar sheets and have previously been proposed to differ in their methyl group orientations in B. mori silk fibers (Carissimi et al., Polymers 12:1 (2020); Asakura et al., Macromolecules 48:28 (2015)). These assignments follow the known relationship between band position and planarity of β-sheets (Kubelka and Keiderling, Journal of the American Chemical Society 123:12048 (2001). Bands at ˜1680 and ˜1669 cm⁻¹ were assigned to other secondary structures and that at 1599 cm⁻¹ was assigned to side chains (Barth, Progress in Biophysics and Molecular Biology 74:141 (2000)) as A₁₅-A₁₄ (and all other constructs) contain 2.3% Glu and 1.4% Arg. The areas of the component bands were divided by the total fitted area of all bands assigned to amide I vibrations (excluding the side chain band) to calculate the relative secondary structure content.

### NMR-Spectroscopy

The solid-state NMR spectra of uniformly ¹³C, ¹⁵N-labeled A₁₅-A₁₄ and (A₃I)₃-A₁₄ fibers were recorded on a Bruker Avance III HD NMR spectrometer equipped with a 3.2 mm ¹H/¹³C/¹⁵N E-free magic-angle spinning (MAS) probe. The sample temperature was set to 277 K. The MAS frequency was 12.5 kHz. 1D ¹H-¹³C cross-polarization (CP) and 2D dipolar assisted rotational resonance (DARR) experiments were acquired using a forward and back CP from ¹H to ¹³C with a linear ramp from 49.0 to 61.2 kHz on 1H and constant ¹³C radiofrequency-field amplitude at 80.5 kHz as well as high-power heteronuclear decoupling at 83.3 kHz during acquisition. The CP contact time was 1 ms and the acquisition time was 10 ms. The ¹³C chemical shifts were referenced externally relative to adamantane (at 38.48 ppm relative to TMS). Spectra were processed with Bruker Topspin 4.0.

### Protein Expression Using a Bioreactor

A fed-batch cultivation of E. coli for expression of (A₃I)₃-A₁₄ was performed as previously described for A₁₅-A₁₄ (Schmuck et al., Materials Today 50:16 (2021) . Briefly, a pre-culture of BL21 (DE3) E. coli transformed for overexpressing (A₃I)₃-A₁₄ was grown in LB-medium (50 μg/mL Kanamycin) at 37° C. Once the OD₆₀₀ reached approximately 5, the pre-culture was used to inoculate (100-fold dilution) fresh 250 mL cultivation medium (50 μg/mL Kanamycin, 0.01% antifoam 204) as defined by da Silva and coworkers (da Silva et al., SpringerPlus 2:1 (2013). A Multifors 2 (Infors) equipped with a 0.5 L glass vessel was used to adjust the pH to 7, with 3 M H₃PO₄ and 25% NH₃. The stirrer speed was adjusted automatically between 200 and 1200 rpm to obtain a dissolved relative oxygen level (pO₂) of 30%. Initially the temperature was set to 28° C., until the OD₆₀₀ reached 50 (22 h after inoculation). Then the temperature was reduced to 20° C. before the culture was induced with IPTG to 150 UM. Feeding was initialized automatically 25 h after inoculation, using the cultivation medium with 40% glycerol, indicated by a sudden increase of pO₂, and following an exponential feeding profile assuming a growth rate of μ=0.1 h⁻¹. Thus, the flow rate was varied between 2.8 and 20 mL/h until 125 ml of the feed stock solution were consumed. 20 h after induction the culture was harvested by centrifugation at 4,000×g, the supernatant was discarded, and the cell pellet was re-suspended in 20 mM Tris, pH 8 (20 mL/10 g wet cell pellet) and stored at −20° C.

### Statistics

Data were analyzed on GraphPad prism, using one-way ANOVA or multi variable analyses (correlation matrix with Pearson correlation coefficients) where appropriate. Statistical significance is indicated with asterisks: * p<0.05; ** p<0.01; *** p<0.001; **** p<0.0001

### Example 2—Industrial Scale Wet Spinning

In an industrial scale wet-spinning process a polymer solution is extruded into the coagulation bath via a metal spinneret, which is an arrangement of one or several hundred orifices. Thus, to make the spinning process described in Example 1 suitable for upscaling, the spinnability using other extrusion devices than the glass capillary needs to be considered. The reasons are that the glass capillaries are fragile and sensitive to damage, and they cannot be re-used with ensured integrity of the capillary. In addition, they are time consuming to manufacture and difficult to make with reproducible geometries. To make extrusion with other devices than glass capillaries possible, the spidroin needs to be compatible with devices that have orifices with a variety of diameters but preferably between 30 to at least 200 μm. Here, compatibility with metal extrusion devices exists if the tip is not blocked by the polymer by premature coagulation and if the spidroin solution can be continuously extruded as a fiber.

### Results

As described in the experimental section of Example 1, glass capillaries with an orifice diameter between 25 and 78 μm were used for extruding concentrated spidroin preparations. However, if glass capillaries with a diameter larger than 150 μm were used, extruding concentrated A₁₅-A₁₄ (300 mg/ml) was not possible. Instead of a fiber, the capillary would either be clogged, likely by premature solidification, or the spinning dope would leave the capillary in the form of blobs. In any case, a continuous fiber was not obtained. Surprisingly, this was not the case for the recombinant spider silk protein (A₃I)₃-A₁₄. If the spinning dope was prepared with (A₃I)₃-A₁₄ (300 mg/ml), and extruded through a glass capillary ≥150 μm, then a continuous fiber was obtained without the occurrence of spontaneous clogging of the tip or the formation of blobs.

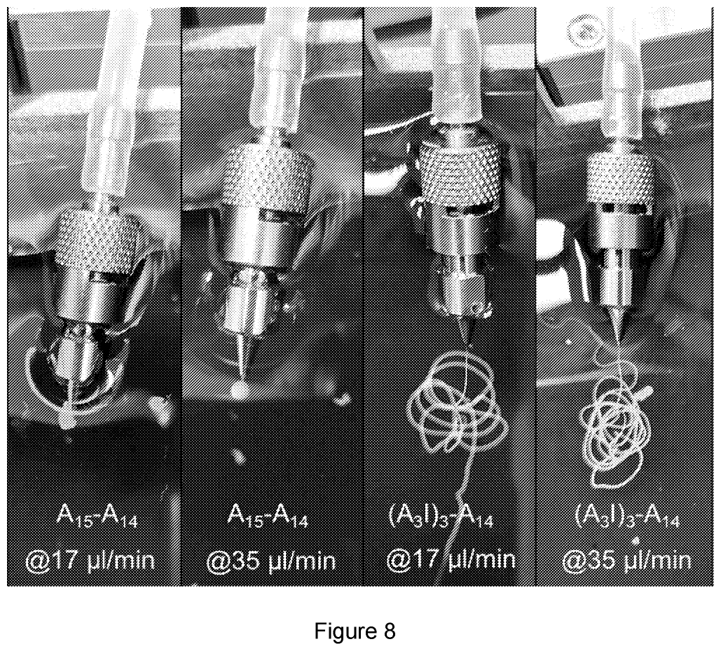

Next, this experiment was repeated but with a metal nozzle, that had an orifice diameter of 150 μm. The extrusion was tested with a flow rate of 17 μl/min and 35 μl/min. Once again, extrusion of A₁₅-A₁₄ into the spinning buffer resulted in clogged tips or blobs (FIG. 8, images on left side), while the extrusion of a concentrated (A₃I)₃-A₁₄ solution was successfully extruded as a silk fiber (FIG. 8, right side). Thus, the recombinant spider silk protein (A₃I)₃-A₁₄ of the invention has significant advantages over A₁₅-A₁₄ not only in mechanical properties as shown in Example 1 but also with respect to the potential for an industrial scale spinning method that relies on metal nozzles and spinnerets for extrusion. Furthermore, the silk fibers produced from recombinant spider silk proteins of the invention can be drawn following spinning.

### Experimental Section

Extrusion with a Glass Capillary ≥150 μm or a Metal Nozzle

A 1 ml syringe with Luer Lok tip (BD, USA) was filled with the concentrated spider silk protein preparation (300 mg/ml) and connected to a glass capillary, as described in Example 1, with a diameter of ≥150 μm. The spinning dope was extruded with a speed of 17 μl/min into a 750 mM acetate buffer, pH 5.0, and the extrusion behavior was observed. As a next step, the syringe was mounted to a Micro-Mate® female Luer to hose end for 1/16 inch (1.5875 mm) to 3/32 inch (2.38125 mm) inner diameter (I.D.) tubing (Cadence Science, Cranston, USA) and connected to a male Luer lock to hose end, for tubing with an I.D. of 1/16- 3/32 inch (Cadence Science, Cranston, USA) via ˜5 cm of silicone tubing with an I.D. of 1.6 mm (667-8441, RS Pro, Gothenburg, Sweden). Finally, the male Luer lock was connected to a Arque metal nozzel (Tecdia, Campbell, USA) with a tip length of 0.3 mm (A-150250000SA-B278) and an inner diameter at the tip of 150 μm. (A₃I)₃-A₁₄ (300 mg/ml) and A₁₅-A₁₄ (300 mg/ml) were extruded through the metal nozzle at 17 or 35 μl/min, and while observing the extrusion behavior.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

1.-18. (canceled)

19. A recombinant spider silk protein comprising an N-terminal (NT) domain, a repetitive region (REP) domain and a C-terminal (CT) domain, wherein
the REP domain comprises a set of domains according to the formula pA₁-pG-pA₂;
pG represents a glycine-rich domain;
pA₁ and pA₂ represent alanine-rich domains;
one of pA₁ and pA₂ is a poly-alanine domain; and
the other of pA₁ and pA₂ is a poly-alanine domain having every third or fourth alanine residue replaced by an isoleucine residue or a valine residue.

20. The recombinant spider silk protein of claim 19, wherein the one of pA₁ and pA₂ comprises an amino acid sequence Aₘ, wherein m is an integer selected within an interval of from 7 up to 18.

21. The recombinant spider silk protein of claim 20, wherein the one of pA₁ and pA₂ consists of an amino acid sequence Aₘ, wherein m is an integer selected within an interval of from 14 up to 16.

22. The recombinant spider silk protein of claim 19, wherein the other of pA₁ and pA₂ is a poly-alanine domain having every fourth alanine residue replaced by an isoleucine residue or a valine residue.

23. The recombinant spider silk protein of claim 22, wherein the other of pA₁ and pA₂ comprises an amino acid sequence selected from the group consisting of (A₃I)ₙAₚ, Aₚ(IA₃)ₙ, (A₃V)ₙAₚ and Aₚ(VA₃)ₙ, wherein n is an integer selected within an interval of from 2 up to 4, p=m−n, and m is an integer selected within an interval of from 8 up to 18.

24. The recombinant spider silk protein of claim 23, wherein the other of pA₁ and pA₂ comprises an amino acid sequence selected from the group consisting of (A₃I)ₙAₚ and Aₚ(IA₃)ₙ, wherein m is an integer selected within an interval of from 10 up to 17.

25. The recombinant spider silk protein of claim 24, wherein the other of pA₁ and pA₂ consists of an amino acid sequence (A₃I)ₙAₚ, wherein n is 3, and m is an integer selected within an interval of from 14 up to 16.

26. The recombinant spider silk protein of claim 19, wherein
the REP domain comprises a set of domains according to a formula selected from the group consisting of pA₁-pG₁-pA₂-pG₂, pG₁-pA₁-pG₂-pA₂ and pG₁-pA₁-pG₂-pA₂-pG₃; and
pG₁, pG₂ and pG₃ represent glycine-rich domains.

27. The recombinant spider silk protein of claim 26, wherein the REP domain comprises a set of domains according to a formula selected from the group consisting of (A₃I)₃A₃-pG₁-A₁₄-pG₂, A₁₅-pG₁-(A₃I)₃A₂-pG₂, pG₁-(A₃I)₃A₃-pG₂-A₁₄, pG₁-A₁₅-pG₂-(A₃I)₃A₂, pG₁-(A₃I)₃A₃-pG₂-A₁₄-pG₃, pG₁-A₁₅-pG₂-(A₃I)₃A₂-pG₃, (A₃V)₃A₃-pG₁-A₁₄-pG₂, A₁₅-pG₁-(A₃V)₃A₂-pG₂, pG₁- (A₃V)₃A₃-pG₂-A₁₄, pG₁-A₁₅-pG₂-(A₃V)₃A₂, pG₁-(A₃V)₃A₃-pG₂-A₁₄-pG₃ and pG₁-A₁₅-pG₂-(A₃V)₃A₂-pG₃.

28. The recombinant spider silk protein of claim 27, wherein the REP domain comprises a set of domains according to a formula selected from the group consisting of (A₃I)₃A₃-pG₁-A₁₄-pG₂, A₁₅-pG₁-(A₃I)₃A₂-pG₂, pG₁-(A₃I)₃A₃-pG₂-A₁₄, pG₁-A₁₅-pG₂-(A₃I)₃A₂, pG₁-(A₃I)₃A₃-pG₂-A₁₄-pG₃ and pG₁-A₁₅-pG₂-(A₃I)₃A₂-pG₃.

29. The recombinant spider silk protein of claim 28, wherein the REP domain comprises a set of domains according to a formula selected from the group consisting of pG₁-(A₃I)₃A₃-pG₂-A₁₄-pG₃ and pG₁-A₁₅-pG₂-(A₃I)₃A₂-pG₃.

30. The recombinant spider silk protein of claim 29, wherein the REP domain comprises a set of domains according to a formula pG₁-(A₃I)₃A₃-pG₂-A₁₄-pG₃.

31. The recombinant spider silk protein of claim 26, wherein each of pG₁, pG₂ and pG₃ comprises an amino acid sequence selected from the group consisting of SEQ ID NO: 49 to 51.

32. The recombinant spider silk protein of claim 31, wherein
pG₁ consists of an amino acid sequence according to SEQ ID NO: 49;
pG₂ consists of an amino acid sequence according to SEQ ID NO: 50; and
pG₃ consists of an amino acid sequence according to SEQ ID NO: 51.

33. The recombinant spider silk protein of claim 19, wherein the REP domain is arranged between the NT domain and the CT domain in the recombinant spider silk protein.

34. The recombinant spider silk protein of claim 33, wherein the recombinant spider silk protein has the general formula (X)-NT-(L1)-REP-(L2)-CT-(Y), wherein X represents an optional N-terminal tag, Y represents an optional C-terminal tag, L1 represents an optional first linker and L2 represents an optional second linker.

35. The recombinant spider silk protein of claim 19, wherein the NT domain is derived from the NT domain of Euprosthenops australis MaSp1.

36. The recombinant spider silk protein of claim 35, wherein the NT domain consists of SEQ ID NO: 53.

37. The recombinant spider silk protein of claim 19, wherein the CT domain is derived from the CT domain of Araneus ventricosus MiSp.

38. The recombinant spider silk protein of claim 37, wherein the CT domain consists of SEQ ID NO: 54.

39. A silk fiber made of a recombinant spider silk protein of claim 19.

40. A synthetic material comprising a silk fiber according to claim 39.

41. A nucleic acid molecule encoding a recombinant spider silk protein of claim 19.

42. An expression vector comprising a nucleic acid molecule according to claim 41.

43. A host cell comprising the expression vector according to claim 42.

44. A method for producing a silk fiber comprising:
extruding a spinning dope comprising a recombinant spider silk protein of claim 19 into an aqueous buffer having an acidic pH to induce polymerization of the recombinant spider silk protein into a silk fiber; and
isolating the silk fiber from the aqueous buffer.


SEQUENCE LISTING (71 sequences)

SEQ ID NO: 1 - protein, 78 aa, synthetic construct
  REGION 1..78: note = REP domain
GRGQGGYGQGSGGNAAAAAAAAAAAAAAAGQGGQGGYGRQSQGAGSAAAAAAAAAAAAAAGSGQGGYGGQGQGGYGQS

SEQ ID NO: 2 - protein, 78 aa, synthetic construct
  REGION 1..78: note = REP domain
GRGQGGYGQGSGGNATATATATATATATAGQGGQGGYGRQSQGAGSATATATATATATATGSGQGGYGGQGQGGYGQS

SEQ ID NO: 3 - protein, 78 aa, synthetic construct
  REGION 1..78: note = REP domain
GRGQGGYGQGSGGNAAATAAATAAATAAAGQGGQGGYGRQSQGAGSAAATAAATAAATAAGSGQGGYGGQGQGGYGQS

SEQ ID NO: 4 - protein, 78 aa, synthetic construct
  REGION 1..78: note = REP domain
GRGQGGYGQGSGGNAVAVAVAVAVAVAVAGQGGQGGYGRQSQGAGSAVAVAVAVAVAVAVGSGQGGYGGQGQGGYGQS

SEQ ID NO: 5 - protein, 78 aa, synthetic construct
  REGION 1..78: note = REP domain
GRGQGGYGQGSGGNAVAVAVAVAVAVAVAGQGGQGGYGRQSQGAGSAAAAAAAAAAAAAAGSGQGGYGGQGQGGYGQS

SEQ ID NO: 6 - protein, 78 aa, synthetic construct
  REGION 1..78: note = REP domain
GRGQGGYGQGSGGNVVVVVVVVVVVVVVVGQGGQGGYGRQSQGAGSAAAAAAAAAAAAAAGSGQGGYGGQGQGGYGQS

SEQ ID NO: 7 - protein, 78 aa, synthetic construct
  REGION 1..78: note = REP domain
GRGQGGYGQGSGGNAAAVAAAVAAAVAAAGQGGQGGYGRQSQGAGSAAAVAAAVAAAVAAGSGQGGYGGQGQGGYGQS

SEQ ID NO: 8 - protein, 78 aa, synthetic construct
  REGION 1..78: note = REP domain
GRGQGGYGQGSGGNAAAVAAAVAAAVAAAGQGGQGGYGRQSQGAGSAAAAAAAAAAAAAAGSGQGGYGGQGQGGYGQS

SEQ ID NO: 9 - protein, 78 aa, synthetic construct
  REGION 1..78: note = REP domain
GRGQGGYGQGSGGNAIAIAIAIAIAIAIAGQGGQGGYGRQSQGAGSAIAIAIAIAIAIAIGSGQGGYGGQGQGGYGQS

SEQ ID NO: 10 - protein, 78 aa, synthetic construct
  REGION 1..78: note = REP domain
GRGQGGYGQGSGGNAAAAAAAAAAAAAAAGQGGQGGYGRQSQGAGSAIAIAIAIAIAIAIGSGQGGYGGQGQGGYGQS

SEQ ID NO: 11 - protein, 78 aa, synthetic construct
  REGION 1..78: note = REP domain
GRGQGGYGQGSGGNAIAAAIAAAIAAAIAGQGGQGGYGRQSQGAGSAIAAAIAAAIAAAIGSGQGGYGGQGQGGYGQS

SEQ ID NO: 12 - protein, 78 aa, synthetic construct
  REGION 1..78: note = REP domain
GRGQGGYGQGSGGNAAAIAAAIAAAIAAAGQGGQGGYGRQSQGAGSAAAIAAAIAAAIAAGSGQGGYGGQGQGGYGQS

SEQ ID NO: 13 - protein, 78 aa, synthetic construct
  REGION 1..78: note = REP domain
GRGQGGYGQGSGGNAAAIAAAIAAAIAAAGQGGQGGYGRQSQGAGSAAAAAAAAAAAAAAGSGQGGYGGQGQGGYGQS

SEQ ID NO: 14 - protein, 78 aa, synthetic construct
  REGION 1..78: note = REP domain
GRGQGGYGQGSGGNAAAAAAAAAAAAAAAGQGGQGGYGRQSQGAGSAAAIAAAIAAAIAAGSGQGGYGGQGQGGYGQS

SEQ ID NO: 15 - protein, 78 aa, synthetic construct
  REGION 1..78: note = REP domain
GRGQGGYGQGSGGNAAIAAIAAIAAIAAAGQGGQGGYGRQSQGAGSAAAAAAAAAAAAAAGSGQGGYGGQGQGGYGQS

SEQ ID NO: 16 - protein, 78 aa, synthetic construct
  REGION 1..78: note = REP domain
GRGQGGYGQGSGGNIAAAAAAIAAAAAAIGQGGQGGYGRQSQGAGSAAAAAAAAAAAAAAGSGQGGYGGQGQGGYGQS

SEQ ID NO: 17 - protein, 6 aa, synthetic construct
  REGION 1..6: note = Hexapeptide
AAAAAA

SEQ ID NO: 18 - protein, 6 aa, synthetic construct
  REGION 1..6: note = Hexapeptide
ATATAT

SEQ ID NO: 19 - protein, 6 aa, synthetic construct
  REGION 1..6: note = Hexapeptide
AAATAA

SEQ ID NO: 20 - protein, 6 aa, synthetic construct
  REGION 1..6: note = Hexapeptide
AVAVAV

SEQ ID NO: 21 - protein, 6 aa, synthetic construct
  REGION 1..6: note = Hexapeptide
VVVVVV

SEQ ID NO: 22 - protein, 6 aa, synthetic construct
  REGION 1..6: note = Hexapeptide
AVAAAV

SEQ ID NO: 23 - protein, 6 aa, synthetic construct
  REGION 1..6: note = Hexapeptide
AIAIAI

SEQ ID NO: 24 - protein, 6 aa, synthetic construct
  REGION 1..6: note = Hexapeptide
AIAAAI

SEQ ID NO: 25 - protein, 6 aa, synthetic construct
  REGION 1..6: note = Hexapeptide
AAIAAI

SEQ ID NO: 26 - protein, 6 aa, synthetic construct
  REGION 1..6: note = Hexapeptide
AAAIAA

SEQ ID NO: 27 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAAAAAAAAAAAAAGQGGQGGYGRQSQGAGSAAAAAAAAAAAAAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 28 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNATATATATATATATAGQGGQGGYGRQSQGAGSATATATATATATATGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 29 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAATAAATAAATAAAGQGGQGGYGRQSQGAGSAAATAAATAAATAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 30 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAVAVAVAVAVAVAVAGQGGQGGYGRQSQGAGSAVAVAVAVAVAVAVGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 31 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAVAVAVAVAVAVAVAGQGGQGGYGRQSQGAGSAAAAAAAAAAAAAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 32 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNVVVVVVVVVVVVVVVGQGGQGGYGRQSQGAGSAAAAAAAAAAAAAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 33 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAVAAAVAAAVAAAGQGGQGGYGRQSQGAGSAAAVAAAVAAAVAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 34 - protein, 347 aa, synthetic construct
  REGION 1..347: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAVAAAVAAAVAAAGQGGQGGYGRQSQGAGSAAAAAAAAAAAAAAGSGQGGYGGQGQGGYGQSGNSSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 35 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAIAIAIAIAIAIAIAGQGGQGGYGRQSQGAGSAIAIAIAIAIAIAIGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 36 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAAAAAAAAAAAAAGQGGQGGYGRQSQGAGSAIAIAIAIAIAIAIGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 37 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAIAAAIAAAIAAAIAGQGGQGGYGRQSQGAGSAIAAAIAAAIAAAIGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 38 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAIAAAIAAAIAAAGQGGQGGYGRQSQGAGSAAAIAAAIAAAIAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 39 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAIAAAIAAAIAAAGQGGQGGYGRQSQGAGSAAAAAAAAAAAAAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 40 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAAAAAAAAAAAAAGQGGQGGYGRQSQGAGSAAAIAAAIAAAIAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 41 - protein, 284 aa, synthetic construct
  REGION 1..284: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAIAAIAAIAAIAAAGQGGQGGYGRQSQGAGSAAAAAAAAAAAAAAGSGQGGYGGQGQGGYGQSGNSVTSGGYGYGTSAAAGAGVAAGSYAGAVNRLSSAEAASRVSSNIAAIASGGASALPSVI

SEQ ID NO: 42 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNIAAAAAAIAAAAAAIGQGGQGGYGRQSQGAGSAAAAAAAAAAAAAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 43 - protein, 14 aa, synthetic construct
  REGION 1..14: note = poly-Ala domain
AAAIAAAIAAAIAA

SEQ ID NO: 44 - protein, 15 aa, synthetic construct
  REGION 1..15: note = poly-Ala domain
AAAIAAAIAAAIAAA

SEQ ID NO: 45 - protein, 14 aa, synthetic construct
  REGION 1..14: note = poly-Ala domain
AAIAAAIAAAIAAA

SEQ ID NO: 46 - protein, 14 aa, synthetic construct
  REGION 1..14: note = poly-Ala domain
AAAVAAAVAAAVAA

SEQ ID NO: 47 - protein, 15 aa, synthetic construct
  REGION 1..15: note = poly-Ala domain
AAAVAAAVAAAVAAA

SEQ ID NO: 48 - protein, 14 aa, synthetic construct
  REGION 1..14: note = poly-Ala domain
AAVAAAVAAAVAAA

SEQ ID NO: 49 - protein, 14 aa, synthetic construct
  REGION 1..14: note = Gly-rich domain
GRGQGGYGQGSGGN

SEQ ID NO: 50 - protein, 17 aa, synthetic construct
  REGION 1..17: note = Gly-rich domain
GQGGQGGYGRQSQGAGS

SEQ ID NO: 51 - protein, 18 aa, synthetic construct
  REGION 1..18: note = Gly-rich domain
GSGQGGYGGQGQGGYGQS

SEQ ID NO: 52 - protein, 78 aa, synthetic construct
  REGION 1..78: note = REP domain
GRGQGGYGQGSGGNAAAAAAAAAAAAAAAGQGGQGGYGRQSQGAGSAAAVAAAVAAAVAAGSGQGGYGGQGQGGYGQS

SEQ ID NO: 53 - protein, 133 aa, synthetic construct
  REGION 1..133: note = Euprosthenops australis MaSp1
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSA

SEQ ID NO: 54 - protein, 121 aa, synthetic construct
  REGION 1..121: note = Araneus ventricosus MiSp
SVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 55 - protein, 332 aa, synthetic construct
  REGION 1..332: note = Spider silk protein
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGRGQGGYGQGSGGNAAAVAAAVAAAVAAAGQGGQGGYGRQSQGAGSAAAAAAAAAAAAAAGSGQGGYGGQGQGGYGQSSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 56 - protein, 338 aa, synthetic construct
  REGION 1..338: note = Spider silk protein
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAVAAAVAAAVAAAGQGGQGGYGRQSQGAGSAAAAAAAAAAAAAAGSGQGGYGGQGQGGYGQSGNSSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 57 - protein, 341 aa, synthetic construct
  REGION 1..341: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGRGQGGYGQGSGGNAAAVAAAVAAAVAAAGQGGQGGYGRQSQGAGSAAAAAAAAAAAAAAGSGQGGYGGQGQGGYGQSSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 58 - protein, 331 aa, synthetic construct
  REGION 1..331: note = Spider silk protein
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGRGQGGYGQGSGGNAAAAAAAAAAAAAAAGQGGQGGYGRQSQGAGSAAAIAAAIAAAIAAGSGQGGYGGQGQGGYGQSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 59 - protein, 337 aa, synthetic construct
  REGION 1..337: note = Spider silk protein
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAAAAAAAAAAAAAGQGGQGGYGRQSQGAGSAAAIAAAIAAAIAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 60 - protein, 340 aa, synthetic construct
  REGION 1..340: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGRGQGGYGQGSGGNAAAAAAAAAAAAAAAGQGGQGGYGRQSQGAGSAAAIAAAIAAAIAAGSGQGGYGGQGQGGYGQSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 61 - protein, 332 aa, synthetic construct; identical to SEQ ID NO: 55
  REGION 1..332: note = Spider silk protein

SEQ ID NO: 62 - protein, 338 aa, synthetic construct; identical to SEQ ID NO: 56
  REGION 1..338: note = Spider silk protein

SEQ ID NO: 63 - protein, 341 aa, synthetic construct; identical to SEQ ID NO: 57
  REGION 1..341: note = Spider silk protein

SEQ ID NO: 64 - protein, 331 aa, synthetic construct
  REGION 1..331: note = Spider silk protein
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGRGQGGYGQGSGGNAAAAAAAAAAAAAAAGQGGQGGYGRQSQGAGSAAAVAAAVAAAVAAGSGQGGYGGQGQGGYGQSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 65 - protein, 337 aa, synthetic construct
  REGION 1..337: note = Spider silk protein
SHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAAAAAAAAAAAAAGQGGQGGYGRQSQGAGSAAAVAAAVAAAVAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 66 - protein, 340 aa, synthetic construct
  REGION 1..340: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGRGQGGYGQGSGGNAAAAAAAAAAAAAAAGQGGQGGYGRQSQGAGSAAAVAAAVAAAVAAGSGQGGYGGQGQGGYGQSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 67 - protein, 346 aa, synthetic construct
  REGION 1..346: note = Spider silk protein
MGHHHHHHMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAAAAAAAAAAAAAGQGGQGGYGRQSQGAGSAAAVAAAVAAAVAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVG

SEQ ID NO: 68 - DNA, 1041 bp, synthetic construct
  misc_feature 1..1041: note = Recombinant spider silk protein
  source 1..1041: mol_type = other DNA
atgggccatcatcatcatcatcatatgagtcacaccactccttggaccaatccgggcttagcggagaacttcatgaacagcttcatgcaaggcttgtcgtcgatgccagggtttacggcatctcagctggatgacatgtccaccattgcacagagtatggtccagtcgatccagagtctcgcagctcaaggtcgcacctcaccgaacaaactgcaagccctgaatatggcgtttgcctcaagcatggcggaaattgctgcctctgaagaaggtggaggttccctgagcacgaaaaccagctctattgcgtcagccatgagcaatgcgtttctgcagacaactggcgttgtgaatcagccgttcatcaacgagatcacgcagcttgtgagcatgtttgcgcaagcaggcatgaacgatgtatccgctgggaattcgggtcgcggacaaggtgggtacggccagggatcgggcggcaacgcggcggcaattgctgcggcgatcgcagcagccattgcggcggcgggtcaaggcggacagggtggttacggtcgtcaatcgcagggcgcgggcagcgcagcagctgccgcagcggccgccgcagcggccgcggctgcaggttccggccaaggtggctatggagggcaggggcagggtgggtatggacagagcggatccgtcacctcaggtggatatggttatggcacgagcgctgccgcgggggcaggcgtggctgccggttcttacgcgggtgccgttaatcgtctctcctccgcggaagcggcatcccgcgtctcaagcaatattgctgcaatcgcatcgggcggcgcgagtgcactacctagcgtgatctcgaacatttattccggtgtagtggcgagcggcgtatcgagcaatgaagcattaatccaggcactcctcgagttactgtccgcgctggttcacgtgctcagcagtgcgagcattggaaacgtcagctcggtgggagtggacagcaccctgaacgttgtccaggattcagtaggtcaatacgttggctaatga

SEQ ID NO: 69 - DNA, 1041 bp, synthetic construct
  misc_feature 1..1041: note = Recombinant spider silk protein
  source 1..1041: mol_type = other DNA
atgggccatcatcatcatcatcatatgagtcacaccactccttggaccaatccgggcttagcggagaacttcatgaacagcttcatgcaaggcttgtcgtcgatgccagggtttacggcatctcagctggatgacatgtccaccattgcacagagtatggtccagtcgatccagagtctcgcagctcaaggtcgcacctcaccgaacaaactgcaagccctgaatatggcgtttgcctcaagcatggcggaaattgctgcctctgaagaaggtggaggttccctgagcacgaaaaccagctctattgcgtcagccatgagcaatgcgtttctgcagacaactggcgttgtgaatcagccgttcatcaacgagatcacgcagcttgtgagcatgtttgcgcaagcaggcatgaacgatgtatccgctgggaattcgggccgcggtcaaggtggatatggacagggcagtggcggcaatgcagcagctgcagcggccgccgccgccgcagcggcagctgccgccggtcaagggggccagggcggatatgggcggcagtcgcaaggcgccggaagtgctgcagctattgccgccgcgatcgcggcggcgattgcggcagggagtggccagggaggctacggcgggcagggccagggtggttacggacagagcggatccgtcacctcaggtggatatggttatggcacgagcgctgccgcgggggcaggcgtggctgccggttcttacgcgggtgccgttaatcgtctctcctccgcggaagcggcatcccgcgtctcaagcaatattgctgcaatcgcatcgggcggcgcgagtgcactacctagcgtgatctcgaacatttattccggtgtagtggcgagcggcgtatcgagcaatgaagcattaatccaggcactcctcgagttactgtccgcgctggttcacgtgctcagcagtgcgagcattggaaacgtcagctcggtgggagtggacagcaccctgaacgttgtccaggattcagtaggtcaatacgttggctaatga

SEQ ID NO: 70 - DNA, 1041 bp, synthetic construct
  misc_feature 1..1041: note = Recombinant spider silk protein
  source 1..1041: mol_type = other DNA
atgggccatcatcatcatcatcatatgagtcacaccactccttggaccaatccgggcttagcggagaacttcatgaacagcttcatgcaaggcttgtcgtcgatgccagggtttacggcatctcagctggatgacatgtccaccattgcacagagtatggtccagtcgatccagagtctcgcagctcaaggtcgcacctcaccgaacaaactgcaagccctgaatatggcgtttgcctcaagcatggcggaaattgctgcctctgaagaaggtggaggttccctgagcacgaaaaccagctctattgcgtcagccatgagcaatgcgtttctgcagacaactggcgttgtgaatcagccgttcatcaacgagatcacgcagcttgtgagcatgtttgcgcaagcaggcatgaacgatgtatccgctgggaattcgggccgcggtcaaggtggatatggacagggcagtggcggcaatgcagcagctgtggcggccgccgtagccgcagcggtcgctgccgccggtcaagggggccagggcggatatgggcggcagtcgcaaggcgccggaagtgctgcagctgcggccgccgcggcggcggcggcggctgcggcagggagtggccagggaggctacggcgggcagggccagggtggttacggacagagcggatccgtcacctcaggtggatatggttatggcacgagcgctgccgcgggggcaggcgtggctgccggttcttacgcgggtgccgttaatcgtctctcctccgcggaagcggcatcccgcgtctcaagcaatattgctgcaatcgcatcgggcggcgcgagtgcactacctagcgtgatctcgaacatttattccggtgtagtggcgagcggcgtatcgagcaatgaagcattaatccaggcactcctcgagttactgtccgcgctggttcacgtgctcagcagtgcgagcattggaaacgtcagctcggtgggagtggacagcaccctgaacgttgtccaggattcagtaggtcaatacgttggctaatga

SEQ ID NO: 71 - protein, 8 aa, synthetic construct
  REGION 1..8: note = His-tag
MGHHHHHH